(19) United States
(12) Patent Application Publication
(10) Pub. No.: US 2025/0345534 A1
(43) Pub. Date: Nov. 13, 2025

(54) AN ELECTRONIC DEVICE FOR CHARACTERIZING AND/OR MONITORING AN OPERATION OF AN INHALER DEVICE, RELATED SYSTEM AND METHOD

(71) Applicant: SONOHALER APS, Kongens Lyngby (DK)

(72) Inventors: Adam BOHR, Kongens Lyngby (DK); Troels TREBBIEN, Kongens Lyngby (DK); Benjamin EJLERTSEN, Kongens Lyngby (DK)

(73) Assignee: SONOHALER APS, Kongens Lyngby (DK)

(21) Appl. No.: 18/859,710

(22) PCT Filed: Apr. 25, 2023
(86) PCT No.: PCT/EP2023/060788

§ 371 Date: Oct. 24, 2024

(30) Foreign Application Priority Data
Apr. 25, 2022 (EP) 22169720.4

(51) Int. Cl. A61M 15/00

(52) U.S. Cl. CPC A61M 15/008; A61M 2205/14; A61M 2205/3334; A61M 2205/3375; A61M 2205/502

(57) ABSTRACT

An electronic device for characterizing and/or monitoring an operation with an inhaler device is disclosed. The electronic device comprises a memory, an interface and a processor comprising predictor circuitry configured to operate according to a prediction model. The processor is configured to obtain operation data (such as operation data), where the operation data is indicative of an audio signal representing an operation with the inhaler device. The processor is configured to determine, based on the operation data, using the predictor circuitry, a predicted operation parameter indicative of a prediction of an operation (such as inhalation flow and/or an exhalation flow) with the inhaler device. The processor is configured to determine, based on the predicted operation parameter, an operation representation.

The present disclosure pertains to the field of electronic devices, and in particular to electronic devices for characterizing and/or monitoring an operation of an inhaler device, related systems and related methods.

### BACKGROUND

Medication inhalers are diverse in their configuration and operation and many users have difficulties using them correctly and knowing whether they used a medication inhaler correctly. Currently, patients are left to themselves to learn and assess their inhalation technique with inhalers aside from a possible initial demonstration by their practitioner. Poor inhalation technique is likely to results in unsatisfactory treatment with any inhaler medication. Poor inhalation can be related to too low or too high inhalation flow, varying (such as fluctuating) inhalation flow, too short inhalation duration, poor coordination (such as poor coordination of an activation and/or release of a medication dose and inhalation), wrong usage of inhaler etc. Wrong usage of the inhaler may for example comprise inhaling from the wrong end of the inhaler, exhaling into the inhaler instead of inhaling, inhaling with too low inhalation flow in the beginning of a medication intake and then ending the medication intake with a too high inhalation flow, and/or having pauses in the inhalation during a medication intake. Adherence, such as lack of adherence, is another major problem for people with asthma and results in unnecessary hospitalization events and incurs great costs to the healthcare system and to society. Adherence can be improved but encouraging users to use their inhaler regularly and remind them to use the inhaler in case they forget to take it. Adherence is mainly related to control inhalers for asthma, typically anti-inflammatory medication that serves as a prophylactic treatment to prevent exacerbations and other undesirable events.

### SUMMARY

There is currently a lack of simple, accurate and convenient technologies for monitoring an operation (such as an inhalation) using inhalers (such as inhaler devices). There is also a lack in technologies that can directly interpret and/or determine the performance of an operation (such as an inhalation) of an inhaler device. Also, there is no existing technology that can determine how and to what degree a medication dose was taken by the user.

Accordingly, there is a need for electronic devices for characterizing and/or monitoring an operation of an inhaler device, systems for characterizing and/or monitoring an operation of an inhaler device, and methods for characterizing and/or monitoring an operation of an inhaler device, which may mitigate, alleviate, or address the shortcomings existing and may provide improved characterization and/or monitoring of an operation of an inhaler device with improved feedback which is more intelligible for the user and with improved accuracy and precision.

An electronic device for characterizing and/or monitoring an operation of an inhaler device is disclosed. The electronic device comprises a memory, an interface and a processor comprising predictor circuitry configured to operate according to a prediction model. The processor is configured to obtain operation data (such as operation data), where the operation data is indicative of an audio signal representing an operation of the inhaler device. The processor is configured to determine, based on the operation data, using the predictor circuitry, a predicted operation parameter indicative of a prediction of an operation (such as an operation, an activation, and/or a coordination) of the inhaler device. The processor is configured to determine, based on the predicted operation parameter, an operation representation. Optionally, the processor is configured to output, via the interface, the operation representation.

Further, a system for characterizing and/or monitoring an operation of an inhaler device is disclosed. The system comprises the inhaler device and an electronic device as disclosed herein.

Further, a method, for characterizing and/or monitoring an operation of an inhaler device is disclosed. The method comprises obtaining operation data, where the operation data is indicative of an audio signal representing an operation of the inhaler device. The method comprises determining, based on the operation data, using the predictor circuitry, a predicted operation parameter indicative of a prediction of an operation of the inhaler device. The method comprises determining, based on the predicted operation parameter, an operation representation. The method comprises outputting, via the interface, the operation representation.

The disclosed electronic device, related method, and system may provide improved characterization and/or monitoring of an operation of an inhaler device with improved accuracy and precision. In other words, the present disclosure may provide improved audio-based characterization and/or monitoring of an inhalation and/or an exhalation with an inhaler device with improved accuracy and precision. It may be appreciated that the present disclosure may provide improved feedback on an operation of an inhaler device, the feedback being more intelligible for the user. The present disclosure may provide an improved prediction of operation parameters, such as an improved prediction of an operation when using an inhaler device. For example, by providing the operation representation the present disclosure may improve the visualization and/or the intelligibility to a user of an operation that the user has performed with an inhaler device. The operation representation may therefore provide information about an operation performance e.g., based on an inhalation parameter, an inhaler device status parameter, an activation parameter, a coordination parameter, and/or a container status parameter. In turn, the present disclosure may provide a faster and more customized feedback to a user after an operation of an inhaler device.

It may be appreciated that the present disclosure provides characterization and/or monitoring of operations with inhaler devices, for example to ensure correct dosing of a medicament when using an inhaler device and track adherence of a user by providing the operation representation. The adherence of a user may be tracked e.g., over a week, a month, and/or a year.

An advantage of the present disclosure is that it may be possible to directly interpret and/or determine the performance of the operations with an inhaler device, based on one or more predicted operation parameters of an operation, for example including an inhalation parameter, an inhaler device status parameter, an activation parameter, and/or a container status parameter. Furthermore, the present disclosure provides the possibility to determine how and to what degree a medication dose was taken by the user, for instance by recognizing if the inhalation was performed by a person and recognizing if a container is emptied. In other words, it may be possible to determine whether a medication intake of a user was successful or not. It may be appreciated that it is possible to recognize whether an inhalation was shallow or deep, continuous or interrupted, smooth or fluctuating, increasing or decreasing, based on the inhalation data, for example based on an inhalation flow pattern. Further it is possible to recognize the likelihood that a medication dose was outputted (such as emitted from an inhaler device), inhaled by a user, and/or deposited (e.g., in the lungs of the user).

Furthermore, an advantage of the present disclosure is that it may be possible for a user of an inhalation device to monitor a status of the inhaler device, such as determining whether the inhaler device needs to be replaced and/or needs maintenance.

Furthermore, an advantage of the present disclosure is that it may be possible for a user of an inhalation device to monitor a status of the container of the inhaler device, such as determining whether the container of the inhaler device needs to be replaced, the container is not properly mounted, the content of the container and/or the type of container.

Further, an advantage of the present disclosure is that the electronic device and the system are more versatile and may be used by any user taking medication with an inhaler device without the need for a healthcare person monitoring the operation of the user.

For example, the present disclosure may provide for training of a user, e.g., by instructing and/or guiding the user through an operation, such as an inhalation. This may for example be useful when a user starts using a new type of inhaler device.

Usually, users are taking the medication with an inhaler device alone at home without help or monitoring from a health care professional. Many patients also do not get proper training when they start using an inhaler device and/or change to a new type of inhaler device. It may be appreciated that the present disclosure provides for remote monitoring of a user of an inhaler device (such as a patient) by a healthcare professional. This enables a more quantitative and informative management for the healthcare professional of their patients and provides more empowerment to patients to take their medication correctly.

Another advantage of the present disclosure is that by using inhalation data indicative of an audio signal (e.g., sound-based) to assess inhalation flow compared with using electronic flow sensors is that it may be possible to obtain a very high sampling rate and temporal resolution of the measurements with sound. An electronic flow sensor may for example comprise an electronic flow meter, such as one or more of a cup anemometer, a pitot tube flow meter, a hot wire flow meter, and a vane flow meter. An electronic flow sensor may be seen as an electronic sensor with an air flow meter. For example, it may be possible to obtain a large number of inhalation flow values per second which can be useful in evaluating dynamic parameters in an inhalation, where the inhalation flow rate can change a lot from one fraction of a second to the next fraction. Further, it may be appreciated that by using inhalation data indicative of an audio signal (e.g., sound-based), the prediction model may be improved over time. This is not possible with inhalation flow rate measurements using non-acoustic sensors. Another advantage is that inhalation data indicative of an audio signal (e.g., sound-based) may also capture unexpected events (something happening in the background of the inhalation) which may also be used to troubleshoot an unsuccessful measurement and/or be used for root cause analysis on a defect of the inhaler device, the container, the prediction model, and/or a microphone of the electronic device.

Further, an advantage of the present disclosure is that the electronic device is more versatile and may be able to characterize and/or monitor an operation performed with any inhaler device.

### BRIEF DESCRIPTION OF THE DRAWINGS

The above and other features and advantages of the present disclosure will become readily apparent to those skilled in the art by the following detailed description of examples thereof with reference to the attached drawings, in which:

FIG. 1 schematically illustrates an exemplary system for characterizing and/or monitoring an operation of an inhaler device according to the present disclosure, comprising an inhaler device and an electronic device according to the present disclosure,

FIGS. 2A-B are flow diagrams of an exemplary method according to the present disclosure,

FIGS. 3A-3B show an example scenario of characterization and/or monitoring of an operation of an inhaler device where the technique disclosed herein is applied,

FIGS. 4A-4D show an example scenario of characterization and/or monitoring of an operation of an inhaler device where the technique disclosed herein is applied,

FIGS. 5A-5D show an example scenario of characterization and/or monitoring of an operation of an inhaler device where the technique disclosed herein is applied,

FIGS. 6A-6D show an example scenario of characterization and/or monitoring of an operation of an inhaler device where the technique disclosed herein is applied,

FIGS. 7A-7B show an example scenario of characterization and/or monitoring of an operation of an inhaler device where the technique disclosed herein is applied,

FIGS. 8A-8B show an example scenario of characterization and/or monitoring of an operation of an inhaler device where the technique disclosed herein is applied,

FIGS. 9A-9B show an example scenario of characterization and/or monitoring of an operation of an inhaler device where the technique disclosed herein is applied, and

FIGS. 10A-10B show an example scenario of characterization and/or monitoring of an operation of an inhaler device where the technique disclosed herein is applied.

### DETAILED DESCRIPTION

Various examples and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the examples. They are not intended as an exhaustive description of the disclosure or as a limitation on the scope of the disclosure. In addition, an illustrated example needs not have all the aspects or advantages shown. An aspect or an advantage described in conjunction with a particular example is not necessarily limited to that example and can be practiced in any other examples even if not so illustrated, or if not so explicitly described.

The figures are schematic and simplified for clarity, and they merely show details which aid understanding the disclosure, while other details have been left out. Throughout, the same reference numerals are used for identical or corresponding parts.

An electronic device for characterizing and/or monitoring an operation of an inhaler device is disclosed. In other words, the electronic device may be configured to characterize and/or monitor an operation with an inhaler device, such as an operation performed with the inhaler device. In other words, the electronic device may be configured to characterize and/or monitor an operation performed by a user when using an inhaler device. The electronic device may be configured to characterize and/or monitor an operation performed with an inhaler device, such as an operation of the inhaler device by a user. An operation may be seen as an operation and/or a procedure of the inhaler device.

An inhaler device may be seen as an inhaler device for inhaling medication. The inhaler device may be seen as a sound generating inhaler, such as an inhaler with acoustic amplifier. In other words, the inhaler device may be an acoustic inhaler device. The inhaler device may comprise an acoustic amplifier in the form of a whistle. The inhaler device may be configured to provide a flow-dependent sound frequency profile, a flow dependent sound amplitude profile, and/or a flow dependent sound energy profile. The inhaler device may alternatively be an inhaler without acoustic amplifier. The inhaler device may comprise different type of inhaler devices, such as powder based inhalers (dry powder inhaler), gas-based inhalers (such as metered dose inhaler and/or propellant-based inhaler), and/or nebulizer atomization based inhalers. The inhaler device may further comprise a single dose inhaler or multidose inhaler. The inhaler device may comprise an add-on container (such as a capsule and/or canister) comprising the medication and/or an integrated medication container (such as capsule, canister, and/or storage) integrated in the inhaler device.

In one or more example electronic devices, the electronic device is a user equipment device.

In one or more example electronic devices, the electronic device is a server device.

The electronic device may comprise a user equipment device and/or a server device. The electronic device may be configured to operate on a user equipment device and/or a server device. In other words, the electronic device may be configured to act as a server device and/or a user equipment device. A user equipment device may for example be or comprise a mobile phone, such as a smartphone, a smart-watch, smart-speakers, a tablet, a computer, such as a laptop computer or PC, or a tablet computer. In other words, the electronic device may for example be a user device, such as a mobile phone or a computer, configured to perform a characterization and/or monitoring of an operation with an inhaler device. A server device may be configured on a cloud, such as a cloud network. Different operations configured to be performed by the electronic device and/or the system as disclosed herein may be performed at different devices, such as at the electronic device and/or at the server device.

The electronic device comprises a memory, an interface and one or more processors comprising predictor circuitry configured to operate according to a prediction model. In other words, the electronic device comprises one or more processors comprising a predictor engine configured to operate according to a prediction model.

The prediction model may for example comprise or make use of a neural network, artificial intelligence, deep learning, and/or machine learning.

In one or more example electronic devices, the prediction model comprises model layers including an input layer, one or more intermediate layers, and an output layer for provision of the predicted operation parameter. In one or more example electronic devices, the prediction model may be seen as a machine learning model. In one or more example electronic devices, the prediction model comprises a neural network. In one or more example electronic devices, the prediction model comprises neural network layers including an input layer, one or more intermediate layers, and an output layer for provision of the predicted operation parameter. In other words, the input layer, the one or more intermediate layers, and/or the output layer may be seen as layers of a machine learning model such as layers of a neural network. The one or more intermediate layers may be considered as hidden layers (such as hidden features). The one or more intermediate layers may include a first intermediate layer.

A model as referred to herein (such as the prediction model) may be seen as a model and/or a scheme and/or a mechanism and/or a method configured to provide, based on operational data (such as an audio signal and/or the operation data) and/or a previous model, one or more predicted operation parameters. A model as referred to herein (such as the prediction model) may be based on the same model architecture. A model architecture may be based on a neural network, such as comprising one or more different type of layers and/or number of layers. A model architecture may be seen as configuration of a model, such as comprising one or more parameters of a model.

In one or more example electronic devices, the model as referred to herein may be stored on a non-transitory storage medium (for example, on the memory of the electronic device). The model may be stored on a non-transitory storage medium of the electronic device being configured to execute the model. In one or more example electronic devices, the model may comprise model data and or computer readable instructions (for example based on operation data and/or audio signal, such as historical operation data). The model data and/or the computer readable instructions may be used by the electronic device and/or the server device. The model (such as model data and/or the computer readable instructions) may be used by the server device and/or the electronic device to determine predicted operation parameters and operation representations. In other words, the model (such as model data and/or the computer readable instructions) may be used by the server device and/or the electronic device to determine one or more parameters and/or features as described herein, such as operation parameter and/or features.

In one or more example electronic devices, the predictor circuitry comprises a neural network module configured to operate according to a neural network.

In one or more example electronic devices, the neural network is a deep neural network, such as a classification neural network configured to operate according to a classification model. In other words, the determination of the predicted operation parameter may comprise to apply a classification model to the operation data.

In one or more example electronic devices, the predictor circuitry comprises a regressor module configured to operate according to a regression model.

The prediction model may be based on a neural network (such as a convolutional neural network, a deep learning neural network, a recurrent neural network, and/or a combined learning circuitry). The predictor circuitry may be configured to determine (and optionally identify) one or more patterns in existing data (operation data, audio signal(s), sound patterns, and/or predicted operation parameters) in order to facilitate making predictions for subsequent predicted operation parameters. For example, the prediction circuitry may be configured to determine (such as recognize) an operation pattern, such as an inhalation pattern, an activation pattern, a coordination pattern, an inhaler status pattern, and/or a container status pattern, based on plot of peak sound frequency over time. Additional prediction models may be generated to provide substantially reliable predictions of inhalation parameters of a prediction of an operation.

The predictor circuitry (such as the neural network module and/or the regressor module) may be configured to operate according to a machine learning scheme configured to determine a rule or a pattern or a relation that maps inputs to outputs, so that when subsequent novel inputs are provided the predictor circuitry may, based upon the rule, pattern or relation, accurately predict the correct output. In one or more embodiments, the prediction model may first extract one or more features from input operation data, such as by using signal processing methods (such as filters), statistics of the signals (such as mean, max, median, and/or quantile), and/or results from unsupervised learning methods (such as dimension reduction methods, clustering, and/or auto-encoder). The one or more features may then be fed into a regression and/or classification model that is trained using machine learning techniques.

In one or more example electronic devices, the processor is configured to train and/or update the prediction model based on one or more of: the operation data and the predicted operation parameter. In one or more embodiments, the processor may be configured to train and/or update the prediction model based on the outcome of the operation representation (for example, by comparing the predicted operation parameter and known inhalation parameters). The prediction model that the predictor circuitry operates according to, may be trained and/or updated (such as retrained or finetuned). The training of the prediction model may be a supervised learning setup, where the operation data in the input data and the network quality data can be labelled. The prediction model or changes to the prediction model may be based on new data, such as new sensor data, and/or new prediction data.

The processor is configured to obtain operation data, where the operation data is indicative of an audio signal (such as audio data) representing an operation with the inhaler device. In other words, the operation data may be based on an audio signal representing an operation with the inhaler device. For example, the operation data may be based on an audio signal from an operation, such as an operation and/or procedure performed by a user with the inhaler device. In other words, the operation data may represent an inhalation operation and/or one or more operations in relation to an inhalation performed by a user of the inhaler device. For example, the operation data may represent and/or be indicative of one or more operations of an inhaler device before and/or after an inhalation and/or an exhalation. The operation data may represent and/or be indicative of one or more operations of an inhaler device before and/or after a medication intake by a user with the inhaler device. The user may be a user of the inhaler device taking a medication dose with the inhaler device. In other words, the user may perform one or more operations, such as a series of operations, when performing a medication intake with the inhaler device, such as performing an inhalation, an exhalation, and/or an activation of the inhaler device. To obtain operation data may comprise that the processor is configured to determine, retrieve, generate, and/or receive the operation data. The operation data may be seen as and/or based on an audio recording of an operation, such as an inhalation operation, performed by a user with the inhaler device. In other words, the operation data may be seen as and/or based on an audio recording of a sound sequence of an operation performed by a user with the inhaler device, such as a sound produced by an inhalation, an exhalation, an amplifier, an activation (such as an actuation) of the inhaler device. The operation data may be based on sound data obtained by the electronic device, such as using the processor, from the inhaler device.

In one or more example electronic devices, the audio signal has a flow dependent sound frequency profile. In other words, the inhaler device may be configured to generate a flow-dependent sound frequency profile that the audio signal is based on. For example, the inhaler device, such as acoustic inhaler device, may be configured to generate an audio signal having a flow dependent sound frequency profile. The inhaler device may comprise an acoustic amplifier, such as a whistle, configured to generate an audio signal comprising a flow-dependent sound frequency profile. For example, the inhaler device comprises a hole tone whistle configured to generate an audio signal comprising a flow-dependent sound frequency profile. In other words, the audio signal, such the sound produced by the inhaler device, may comprise a sound signature when performing an operation with the inhaler device. For example, the audio signal, such the sound produced by the inhaler device, may comprise a distinct sound frequency profile for each level of air flow when using the inhaler device. It may be appreciated that the higher the inhalation flow is the louder (amplitude) the sound produced by an inhaler device becomes. This can also be translated as sound energy. For example, by using an inhaler device comprising an acoustic amplifier (such as a whistle) having a substantially linear relationship between sound frequency and flow rate the electronic device, such as using the predictor circuitry, may determine a predicted inhalation parameter indicative of an inhalation flow with the inhaler device, e.g., based on the sound frequency profile of the audio signal generated by the inhaler device.

By using the sound frequency sound profile, a more robust prediction of inhalation flow may be achieved as it is independent of distance from the inhaler device.

This is an advantage compared to inhaler devices having an acoustic amplifier being harmonic devices, or other types of inhaler devices that do now show such a clear relationship between sound frequency and flow rate.

In one or more example electronic devices, the electronic device comprises one or more microphones for obtaining the audio signal. For example, the electronic device may comprise a mobile phone comprising one or more microphones for obtaining the audio signal. In other words, the electronic device comprises one or more microphones configured to generate and/or provide the audio signal based on a sound generated by the inhaler device and/or the user of the inhaler device, such as the acoustic inhaler device. The one or more microphones may have a sampling rate of at least 50 kHz. By having a sampling rate of at least 50 kHz it may ensure that sound from the inhaler device is captured at high resolution and that an electronic device according to the present disclosure, e.g., with a sound-based inhalation flow meter, provides a high temporal resolution for the predicted inhalation parameter. This is an advantage because of the high sampling rate and continuity of the inhalation data, e.g., compared with sensor based technology, such as electronic flow sensors.

The processor is configured to determine, based on the operation data, using the predictor circuitry, a predicted operation parameter indicative of a prediction of an operation of the inhaler device. A predicted operation parameter may be seen as a predicted physiological factor indicative of a prediction of an operation with the inhaler device. An operation as disclosed herein may be seen as one or more operations, such as comprising a first operation, a second operation, and/or a third operation. An operation as disclosed herein may comprise a series of operations. In other words, the processor is configured to determine, based on the operation data, using the prediction model, a predicted operation parameter indicative of a prediction of an operation of the inhaler device. In one or more example electronic devices, the processor is configured to determine, based on the operation data, using the predictor circuitry, a predicted operation parameter indicative of an operation of the inhaler device, such as an inhalation, an exhalation, and/or an activation of the inhaler device. Determining a predicted operation parameter indicative of a prediction of an operation may comprise extracting one or more operation features from the audio signal and/or the operation data. In other words, determining a predicted operation parameter indicative of a prediction of an operation may be based on one or more features extracted from the audio signal and/or the operation data. In one or more example electronic devices, the processor is configured to determine the predicted operation parameter based on one or more features extracted from the audio signal and/or the operation data. For example, when the audio signal has a flow dependent sound frequency, the processor may be configured to extract a sound frequency from the audio signal, and to determine a predicted operation parameter based on the extracted sound frequency. In other words, the audio signal may have a flow dependent sound frequency profile (e.g., sound spectral profile) and the processor may be configured to extract one or more sound frequencies, such as one or more sound frequency peaks, from the audio signal, and to determine a predicted operation parameter based on the extracted one or more sound frequencies. For example, the inhalation data, such as the audio data, may comprise for each inhalation and/or exhalation multiple sound frequency peaks.

The one or more operation features may for example comprise one or more of: an operation phase (such as a phase of an operation of the user when taking medication with the inhaler device), an activation feature, an inhaler device feature, a container feature, an inhalation flow feature, an amplitude feature, a time feature (such as duration feature), an inhalation volume feature, and a flow acceleration feature. An operation phase may comprise one or more of: a preparation phase (such as preparation of container, e.g., pinching capsule), an introductory inhalation phase, an intermediate inhalation phase, an ending inhalation phase, an exhalation phase, and an activation (such as actuation) phase.

In one or more example electronic devices, the determination of the predicted operation parameter comprises to determine one or more of: an inhalation parameter, an inhaler device status parameter, an activation parameter, a coordination parameter, and a container status parameter. In other words, the electronic device, such as the processor, is configured to determine, e.g., using the predictor circuitry, one or more of: an inhalation parameter, an inhaler device status parameter, an activation parameter, a coordination parameter, and a container status parameter. An inhalation parameter may be seen as a parameter indicative of an inhalation (such as an inhalation operation) performed with the inhaler device, such as an inhalation performed by a user of the inhaler device. An inhalation parameter may be seen as an inhalation characteristic. An inhalation parameter may comprise one or more inhalation flows, such as an inhalation flow over time.

For example, the inhalation parameter may comprise an inhalation flow based on operation data indicative of an audio signal representing sound merely from an inhalation by the user and not from an acoustic amplifier (such as whistle) of the inhaler device. The inhalation parameter may comprise one or more of: a duration of an inhalation, an inhalation volume, an average inhalation flow rate, a maximum inhalation flow rate, a minimum inhalation flow rate, median inhalation flow, an inhalation flow acceleration, and an inhalation pattern. In other words, the determination of the inhalation parameter may be based on an audio signal representing an inhalation with the inhaler device.

An inhaler device status parameter may be seen as a parameter indicative of a status of the inhaler device. For example, the inhaler device status parameter may indicate whether the inhaler device is working or not (e.g., not working properly), whether the inhaler device is positioned correctly or not (e.g., not positioned correctly when performing an inhalation), the type of inhaler device, and/or whether the inhaler device needs maintenance or not. In other words, the determination of the inhaler device status parameter may be based on an audio signal representing an inhaler device sound (such as an inhaler device sound signature).

An activation parameter may be seen as a parameter indicative of an activation of the inhaler device, such as an activation of the inhaler device when performing an operation with the inhaler device. An activation may be seen as an activation and/or an actuation of the inhaler device and/or the container of the inhaler device. In other words, an activation parameter may be indicative of an activation of a container of the inhaler device. For example, the activation parameter may indicate whether an activation has been performed or not, whether a medication dose has been outputted by the inhaler device or not. The activation parameter may comprise an activation time (such as timestamp), an activation type, and/or an activation duration. An activation of the inhaler device, such as a way of activating the inhaler device, may depend on the type of inhaler device. For example, for an inhaler device comprising a container being of a canister type, the activation may comprise to press and release the canister. For an inhaler device comprising a container being of a capsule type, the activation may comprise to pinch the capsule to make a hole in the capsule. For an inhaler device being of a nebulizer type, the activation may comprise to turn on a nebulizer. In other words, the determination of the activation parameter may be based on an audio signal representing an activation of the inhaler device.

A coordination parameter may be seen as a parameter indicative of a coordination of one or more operations with the inhaler device, such as an activation of the inhaler device and an inhalation with the inhaler device. For example, the coordination parameter may indicate whether a coordination has been successful or not. The coordination parameter may comprise one or more timestamps for the one or more operations to be coordinated. In other words, the determination of the coordination parameter may be based on an audio signal representing a coordination of one or more operations with the inhaler device.

A container status parameter may be seen as a parameter indicative of a status of the container of the inhaler device. For example, the container status parameter may indicate whether the container of the inhaler device is operational or not, the type of container, whether a container is present in the inhaler device or not, a content status of the container, whether a container was emptied during an inhalation (e.g., if it is a capsule-type container), and/or whether the container is correctly mounted and/or arranged in the inhaler device or not. In other words, the determination of the container status parameter may be based on an audio signal representing a container sound (such as a container sound signature).

In one or more example electronic devices, the operation data may be indicative of a plurality of operations, and the predicted operation parameter may be determined based on a prediction of a combination of operations. For example, the audio signal may be indicative of an inhalation sound and at the same time a capsule rotation sound. It may be appreciated that the predicted operation parameter may be determined based on a detection, identification, and/or classification of an interaction and/or interference between the sounds from a plurality of operations.

In one or more example electronic devices, the determination of the container status parameter comprises to determine whether a container is present in the inhaler device. In other words, the electronic device is configured to determine whether a container is present in the inhaler device. To determine whether a container is present may comprise to determine whether a container (such as canister and/or a capsule) is correctly mounted in the inhaler device. This may be particularly advantageous when using inhaler devices with add-on containers, where the user may have to mount the container on the inhaler device. By determining whether a container is present in the inhaler device, it may be possible to determine whether a container was present when the user performed an operation (such as an inhalation) with the inhaler device. To determine whether a container is present may comprise to determine which type of container is present in the inhaler device. For example, when using a capsule-type inhaler device, the capsule will rotate while performing an inhalation, making a different sound than when using a canister-type inhaler device.

In accordance with the determination that a container is present in the inhaler device, the electronic device is configured to determine a recommendation, such as a first recommendation as disclosed herein. The electronic device may for example, in response to the determination that a container was present, be configured to determine one or more recommendations such as: “A container is mounted on your inhaler device, you should be able to perform a successful inhalation with your inhaler device”, and/or “It was established that a container was mounted on your inhaler device while performing an operation”. Alternatively or additionally, the electronic device may be configured to refrain from determining a recommendation when it is determined that a container is present in the inhaler device. The determination of whether a container is present in the inhaler device or not may be used by the electronic device, such as the processor, to determine whether an operation was successful or not.

In one or more example electronic devices, in accordance with the determination that a container is not present in the inhaler device, the electronic device is configured to determine a recommendation, such as a second recommendation and/or a container recommendation as disclosed herein. The electronic device may for example, in response to the determination that no container was present, be configured to determine one or more recommendations such as: “There is no container mounted on your inhaler device, please mount a container on the inhaler device”, and/or “It was established that no container was mounted on your inhaler device, please verify that a container is mounted and/or that the container is correctly mounted”.

In one or more example electronic devices, the determination of the container status parameter comprises to determine a content of a container of the inhaler device. In other words, the electronic device is configured to determine a content of a container in or of the inhaler device. To determine a content of a container may comprise to determine whether a container (such as canister and/or a capsule) is full, half-full, empty, and/or monitor the emptying of the container during an operation. To determine a content of a container may comprise to determine content of medication left in the container. In one or more example electronic devices, the user may provide an input indicating a type of container of the inhaler device. The determination of a content of a container may be used to determine how a medication dose was outputted during the inhalation, e.g., if a container (such as capsule) emptied completely or not during the inhalation.

By determining a content of a container of the inhaler device, it may be possible to determine whether a medication dose can be outputted and/or was outputted when the user performed an operation (such as an inhalation) with the inhaler device. For example, when using a capsule-type inhaler device, the capsule will rotate while performing an inhalation, making a different sound depending on the weight of the capsule which is changing while the capsule is being emptied during the inhalation.

In one or more example electronic devices, the obtaining of the operation data comprises to obtain, based on the audio signal, sound data of the audio signal, and wherein the determination of the predicted operation parameter is based on the sound data. In one or more example electronic devices, the processor is configured to obtain, based on the audio signal, sound data of the audio signal, e.g., from the inhaler device. In one or more example electronic devices, the processor is configured to determine the predicted operation parameter based on the sound data. In other words, the processor may be configured to extract one or more audio features from the audio signal, where the sound data comprises the one or more audio features. It may be appreciated that the prediction model may be configured to extract one or more audio features from the audio signal to obtain (such as provide) the sound data. The sound data may comprise one or more audio features, such as a sound frequency feature (such as spectrum of sound frequencies for each point in time), an amplitude feature, and/or a time feature. In other words, the sound data may comprise a sound frequency profile over time, and/or an amplitude profile over time. The sound frequency may for example be in the range of 0 to 25 kHz. The sound data may comprise frequency peak changes over time in the course of an operation, such as during an inhalation. In one or more example electronic devices, the obtaining of the sound data comprises to obtain sound data based on image data. In other words, obtaining sound data may comprise to extract one or more audio features from image data, such as image data comprising a spectrogram, e.g., by using image analysis. For example, obtaining sound data may comprise to identify one or more frequency peak changes over time in the course of an operation, such as during an inhalation. For example, obtaining sound data may comprise to identify one or more frequency peak changes over time in a spectrogram.

In one or more example electronic devices, the sound data comprises one or more of: a frequency signature, an amplitude signature, and a duration signature. For example, the predictor circuitry may be configured to classify, identify, and/or detect (such as using the prediction model, e.g., a classification model) a signature, such as a frequency signature, an amplitude signature, and/or a duration signature. A signature, such as a frequency signature, an amplitude signature, and/or a duration signature may for example be classified, identified, and/or detected for the same user and/or across several users. It may for example be possible to classify, identify, and/or detect a condition of a user based on the sound data. In other words, a signature may be indicative of a condition of a user, such as lung condition and/or a respiratory condition. Furthermore, it may be possible to classify, identify, and/or detect an anomaly, such as an anomaly of the inhaler device, an anomaly of the use of the inhaler device, and/or an anomaly of a microphone of the electronic device. A signature may be seen as a classification, an identification and/or a detection of a pattern.

In one or more example electronic devices, the obtaining of the operation data comprises to perform pre-processing of the audio signal. In one or more example electronic devices, the processor may be configured to perform pre-processing of the audio signal to obtain, such as determine, the operation data. In other words, the processor comprises a pre-processing module configured to perform pre-processing of the audio signal. For example, the obtaining of the operation data may comprise to apply one or more filters to the audio signal and/or to transform the audio signal. To apply one or more filters may comprise to apply a low-pass filter and/or a high-pass filter to the audio signal. By applying one or more filters to the audio signal the signal to noise ratio (SNR) may be enhanced. In one or more example electronic devices, the prediction circuitry is configured to perform pre-processing by using the prediction model on the audio signal. In other words, the prediction model may be configured to extract the audio signal representing an operation with an inhaler device from an audio signal (such as audio data) by performing pre-processing, such as by applying one or more filters.

In one or more example electronic devices, the obtaining of the operation data comprises to identify a background noise (such as background sounds) from the audio signal. In one or more example electronic devices, the processor may be configured to identify a background noise from the audio signal. In one or more example electronic devices, the prediction circuitry is configured to identify a background noise by using the prediction model on the audio signal. For example, the prediction model may be pre-trained based on a plurality of background noises in order to be able to identify one or more background noises from the audio signal. In other words, the prediction model may be trained to extract the audio signal representing an operation with an inhaler device from an audio signal (such as audio data) comprising one or more background noises (such as background sounds).

In one or more example electronic devices, the obtaining of operation data comprises to split (such as cut) the audio signal (such as audio data) into a plurality of audio samples. In one or more example electronic devices, the processor is configured to split the audio signal into a plurality of audio samples. For example, the processor may comprise an audio processing module configured to process the audio signal, such as configured to split the audio signal into a plurality of audio samples. In other words, by splitting the audio signal (such as audio data) into a plurality of audio samples it may be possible to divide the audio signal into different phases of the operation. A phase of the operation may for example be an activation of the inhaler device (such as activating a container of the inhaler device to be in a state where the inhaler device can output medication). By splitting up the audio signal into a plurality of audio samples the accuracy of the determination of the predicted operation parameter may be improved since the determination of the predicted operation parameter is based on a shorter audio sample than the complete audio signal of the whole operation. Furthermore, it may be appreciated that the probability that there are several operations occurring during the same audio sample is lower and thereby improving the determination of the predicted operation parameter, such as improving the identification of an operation. In one or more example electronic devices, it may be advantageous to determine the predicted operation parameter based on operation data representing a complete medication intake operation (such as a complete inhalation operation). For example, when determining a container status parameter, such as a container (e.g., capsule and/or canister) emptying over time, the determination of the predicted operation parameter may be based on operation data representing a complete medication intake operation (such as a complete inhalation operation).

In one or more example electronic devices, the determination of the predicted operation parameter is based on one or more audio samples of the plurality of audio samples. In one or more example electronic devices, the processor is configured to determine the predicted operation parameter based on one or more audio samples of the plurality of audio samples. In other words, the predictor circuitry may be configured to provide one or more audio samples of the plurality of audio samples as an input to the prediction model. In other words, the processor (such as the predictor circuitry) may be configured to provide the plurality of the audio samples as an input to the prediction model and keep the structure of the audio signal, such as keep the chronology of the audio samples.

To split the audio signal (such as audio data) into a plurality of audio samples may comprise to split the audio signal at a sample rate in the range of 1 ms to 1000 ms, such as in the range of 5 ms to 100 ms. By splitting the audio signal into a plurality of audio samples, it may be possible to balance between capturing more details about the inhalation and/or the exhalation with shorter audio samples when determining the predicted operation parameter, and flattening out the data by averaging out the audio signal over fewer audio samples, such as averaging out an inhalation flow profile over fewer sample points, when determining the predicted operation parameter. For example, by splitting the audio data into short audio samples in the range of 1 ms to 10 ms it may be possible to determine a predicted operation parameter representative of a specific period of the operation, such as predicted operation parameter representative of a specific event of the operation. By using audio samples in the range of 1 ms to 1000 ms, it may be possible to reduce the risk of confusion for the prediction model since the audio samples are representative of substantially one event of the operation.

In one or more example electronic devices, the obtaining of operation data comprises to shuffle the plurality of audio samples. In one or more example electronic devices, the processor is configured to shuffle the plurality of audio samples. In one or more example electronic devices, the determination of the predicted operation parameter is based on the shuffled audio samples.

In one or more example electronic devices, the processor is configured to determine the predicted operation parameter based on the shuffled audio samples. In other words, the predictor circuitry may be configured to provide one or more of the shuffled audio samples of the plurality of audio samples as an input to the prediction model. In other words, the processor (such as the predictor circuitry) may be configured to provide the plurality of shuffled audio samples as an input to the prediction model. By providing the plurality of shuffled audio samples as an input to the prediction model the prediction model may be trained to determine the predicted operation parameter without having the structure and/or chronology of the audio samples. This may provide a more robust prediction model to determine predicted operation parameters. Further, by splitting (such as cutting) and/or shuffling the audio signal, more audio data is provided, for example to train the prediction model, expand the audio data library, and/or determine predicted operation parameters on smaller audio samples. It may be appreciated that it may be possible to determine a plurality of predicted operation parameters based on the same medication intake (such as inhalation operation).

In one or more example electronic devices, the obtaining of operation data comprises to transform the audio signal to a spectrogram. In one or more example electronic devices, the processor is configured to transform the audio signal to a spectrogram.

In one or more example electronic devices, the determination of the predicted operation parameter is based on the spectrogram. In one or more example electronic devices, the processor is configured to determine the predicted operation parameter based on the spectrogram.

In one or more example electronic devices, the obtaining of operation data comprises to transform the audio signal to a spectrogram and to split the spectrogram into a plurality of spectrogram samples. In one or more example electronic devices, the determination of the predicted operation parameter is based on one or more of the plurality of spectrogram samples.

In one or more example electronic devices, the obtaining of operation data comprises to transform the audio signal to a spectrogram, to split the spectrogram into a plurality of spectrogram samples, and to shuffle the plurality of spectrogram samples. In one or more example electronic devices, the determination of the predicted operation parameter is based on the plurality of shuffled spectrogram samples. By transforming the audio signal to a spectrogram it may be possible to classify one or more events of the operation by using the prediction model. A spectrogram may for example comprise a spectrogram representing frequency over amplitude which may be seen as a still shot of an operation (such as of an inhalation). A spectrogram may for example comprise a spectrogram representing frequency over time which may be seen as a representation of an entire operation (such as of an entire inhalation).

In one or more example electronic devices, the obtaining of the operation data comprises to obtain image data, such as based on the audio signal. In one or more example electronic devices, the processor is configured to obtain image data. In one or more example electronic devices, the determination of the predicted operation parameter is based on the image data. In one or more example electronic devices, the processor is configured to determine the predicted operation parameter based on the image data. In other words, the processor is configured to use the image data as input to the prediction model. In one or more example electronic devices, the processor is configured to determine one or more image features based on the image data. The one or more image features may be indicative of one or more of: an operation phase (such as a phase of an operation of the user when taking medication with the inhaler device), an activation, a container status, an inhaler device status, a coordination, an inhalation flow, an amplitude, time (such as duration), an inhalation volume, and an inhalation flow acceleration.

In one or more example electronic devices, the processor is configured to determine the predicted operation parameter based on the one or more features.

The image data may comprise an image indicative of a sound frequency of the operation. In other words, the image data may for example comprise a plot, such as a graph, e.g., a two dimensional plot, with one or more of the sound frequency, the amplitude, and the energy on the y-axis and the time on the x-axis. For example, the image data may comprise a time lapse of an inhalation and/exhalation. A spectrogram as described above may be seen as image data, such as the spectrogram having the format of an image.

In one or more example electronic devices, the determination of the predicted operation parameter comprises to determine inhalation flow data. Inhalation flow data may for example comprise one or more of the features as described above, such as the one or more extracted features and/or the one or more operation features. The determination of the inhalation flow data may comprise to determine a predicted inhalation flow, such as a predicted inhalation flow over time.

In one or more example electronic devices, the processor is configured to determine, based on the predicted operation parameter, one or more of: a duration of the operation, a coordination of the operation, an inhalation volume, an average inhalation flow, a maximum inhalation flow, a minimum inhalation flow, median inhalation flow, an inhalation flow acceleration, an inhalation pattern, and one or more exhalation parameters. In other words, the predicted operation parameter may comprise one or more of: a duration of the operation, a coordination of the operation(s), an inhalation volume, an average inhalation flow, a maximum inhalation flow, a minimum inhalation flow, median inhalation flow, an inhalation flow acceleration, and one or more exhalation parameters. In other words, an output of the prediction model may comprise one or more of: a duration of the operation, a coordination of the operation, an inhalation volume, an average inhalation flow, a maximum inhalation flow, a minimum inhalation flow, median inhalation flow, an inhalation flow acceleration, and one or more exhalation parameters. In one or more example electronic devices, the processor is configured to determine, based on the operation data and/or the audio signal, one or more of: a duration of the operation, a coordination of the operation, an inhalation volume, an average inhalation flow, a maximum inhalation flow, a minimum inhalation flow, median inhalation flow, an inhalation flow acceleration, and one or more exhalation parameters.

To determine a predicted operation parameter may comprise to determine a duration of the operation. For example, a duration of an activation, a duration of an inhalation, and/or a duration of an exhalation may be determined.

To determine a predicted operation parameter may comprise to determine a coordination of the operation(s). A coordination of the operation (such as operations) may be seen as a coordination between one or more operations performed by a user with the inhaler device. For example, a coordination of the operation may be seen as a coordination of an activation of the inhaler device (such as an actuation) and an inhalation with the inhaler device, such as a start of an inhalation. Depending on the type of inhaler device, the coordination of the operation may be different. For example, when using a spray-type inhaler device, to perform a successful medication intake the user would have to start inhaling first and then activate the inhaler device by actuating the container of the inhaler device to output a medication dose while continuing the inhalation. On the other hand, when using a powder-type inhaler device, to perform a successful medication intake the user would first activate the inhaler device by for example turning an activation mechanism of the inhaler device to output a medication dose (such as powder) and then start the inhalation. The coordination of operation(s) may therefore be different depending on the type of inhaler.

To determine a predicted operation parameter may comprise to determine a pattern, such as an operation pattern, an inhalation pattern, and/or an exhalation pattern. A pattern may be seen as an act, an action, and/or a characteristic that are repeated by the user during one or more operations. A pattern may for example comprise an operation pattern, an inhalation pattern, and/or an exhalation pattern. To determine a pattern, such as an operation pattern, an inhalation pattern, and/or an exhalation pattern, may comprise to identify one or more acts, actions, and/or characteristics that are repeated by the user during one or more operations.

For example, a duration of an activation, a duration of an inhalation, and/or a duration of an exhalation may be determined.

An exhalation parameter may comprise one or more of: an exhalation flow, a duration of exhalation, an exhalation volume, an average exhalation flow, a maximum exhalation flow, an exhalation inhalation flow, median exhalation flow, and an exhalation flow acceleration.

The processor is configured to determine, based on the predicted operation parameter, an operation representation. The operation representation may be seen as a representation indicative of all or part of the operation performed by a user with the inhaler device. In other words, the operation representation may be seen as an inhaler device operation representation. The operation representation may be seen as and/or comprise an evaluation of an operation with the inhaler device, such as an operation evaluation and/or an inhalation evaluation. In other words, the operation representation may be seen as and/or comprise an evaluation of an operation of a user when taking a medication with the inhaler device.

In one or more example electronic devices, the operation representation is indicative of a performance of the operation of the inhaler device. For example, the operation representation may be indicative of a low performance when an inhalation was too low (e.g., based on the sound volume), an inhalation was too short, an inhalation was performed with an empty container, and/or that a coordination of operation(s) was faulty and/or wrong. In other words, a performance of an operation of the inhaler device may be seen as a performance quality of an operation of the inhaler device. For example, a performance of an operation of the inhaler device may be seen as how a user of the inhaler device has performed a certain operation of the inhaler device.

In one or more example electronic devices, the operation representation comprises an operation score indicative of a performance of a user when performing an operation with the inhaler device, such as a performance of a medication intake. The operation representation may indicate whether an operation, an activation, a coordination of operation(s), an inhalation, an exhalation, and/or a medication intake was successful or not. The operation representation may provide information to the user regarding an inhaler device status, such as information regarding maintenance of the inhaler device. The operation representation may provide information to the user regarding a container and/or a container status of an inhaler device, such as information regarding a presence of a container and/or regarding a content of a container. For example, the score may be indicative of a successful inhalation, exhalation, and/or medication intake when the predicted operation parameter satisfies a criterion. For example, the processor may be configured to determine whether the score satisfies a criterion (such as the first criterion). When the score is above or equal to a threshold (such as the first threshold), it may be determined that the score satisfies the criterion (such as first criterion).

In one or more example electronic devices, the operation representation comprises a representation of the predicted operation parameter. For example, the operation representation may comprise a representation of an inhalation flow with the inhaler device, such as a graph or a plot of the inhalation flow over time, a spectrogram and/or the image data as described herein. The operation representation may comprise one or more of: a representation of an inhalation parameter, a representation of an inhaler device status, a representation of an activation, a representation of a container status, a representation of a duration of inhalation, a representation of an inhalation volume, a representation of an inhalation flow profile, a representation of an average inhalation flow, a representation of a maximum inhalation flow, a representation of a minimum inhalation flow, a representation of a median inhalation flow, a representation of an inhalation flow acceleration, and a representation of one or more exhalation parameters. The operation representation may be indicative of the whole operation with the inhaler device, and/or part of the operation with the inhaler device, such as a phase of the operation with the inhaler device.

In one or more example electronic devices, the operation representation is indicative of an evaluation of an inhaler device. In other words, the processor may be configured to determine a state of the inhaler device based on the operation data and/or the audio signal.

An advantage of having an operation representation, e.g. after an inhalation, an exhalation, and/or an operation of the inhaler device has been performed, may be that the user of the electronic device and/or the inhaler device may see or be informed right after the inhalation, such as operation, about his/her performance or the outcome of the operation in relation to the predicted operation parameter. Therefore, when an operation was unsuccessful the user may be aware of it and may be able to repeat the inhalation and/or operation and/or improve his/her performance for the next inhalation and/or operation. Furthermore, the user of the electronic device and/or the inhaler device may get a better feedback on his/her performance or on the outcome of the inhalation and/or the operation. The operation representation may provide a gamification of the users' performances. The operation representation may for example increase the intelligibility of the feedback to the user, e.g. by being able to visualize an inhalation and/or operation of the inhaler device, and further to improve his/her inhalation technique by being able to visualize an improvement of performances. The operation representation may provide feedback regarding the status of a container of the inhaler device, e.g., when a container is not arranged properly and/or a container is empty or close to become empty. The user may therefore reduce the number of unsuccessful operations and/or medication intakes and in any case be informed about an unsuccessful medication intake and/or operation so that the user may repeat the medication intake and/or operation.

Optionally, the processor is configured to output, via the interface, the operation representation.

In one or more example electronic devices, outputting the operation representation may comprise outputting, via the interface of the electronic device, the operation representation. Outputting the operation representation may comprise displaying a user interface indicative of the operation representation. In one or more exemplary electronic devices, outputting the operation representation may comprise outputting, via the interface of the electronic device, a first operation representation, a second operation representation, a third operation representation, etc.

Outputting the operation representation may comprise displaying a user interface indicative of the operation representation.

A user interface may comprise one or more, such as a plurality of, user interface objects. For example, the user interface may comprise one or more user interface objects, such as a first user interface object and/or a second user interface object. A user interface object may refer herein to a graphical representation of an object that is displayed on an interface of the electronic device, such as a display. The user interface object may be user-interactive, or selectable by a user input. For example, an image (e.g., icon), a button, and text (e.g., hyperlink) each optionally constituting a user interface object. The user interface object may form part of a widget. A widget may be seen as a mini-application that may be used by the user. To output the operation representation may comprise to output an operation representation comprising one or more of text (such as a text string) and/or a phrase, a score (such as an evaluation score and/or an operation score), image data (such as one or more images), a spectrogram, and/or a user interface object comprising one or more of the previous. For example, to output the operation representation may comprise to output an operation representation comprising a spectrogram of an inhalation flow profile over time as disclosed herein and an indication of where on the spectrogram the user may improve his/her operation. For example, to output the operation representation may comprise to output an operation representation comprising a score, such as an evaluation score of the operation of the user with the inhaler device. For example, to output the operation representation may comprise to output an operation representation comprising an evaluation of the inhaler device, such as comprising an inhaler device status. For example, to output the operation representation may comprise to output an operation representation comprising an evaluation of an activation of the inhaler device, such as an evaluation of an activation and/or actuation of a container (such as capsule and/or canister) of the inhaler device. For example, to output the operation representation may comprise to output an operation representation comprising an evaluation of a status of a container of the inhaler device.

In one or more example electronic devices, the processor is configured to determine whether the predicted operation parameter satisfies a first criterion. In other words, the processor may be configured to determine whether the predicted operation parameter is above, below, or equal to an inhalation flow threshold and/or is within a certain range indicative of the first criterion. In other words, the predicted operation parameter may satisfy the first criterion when the predicted operation parameter is above or equal to a first threshold. For example, the predicted operation parameter may satisfy the first criterion when the inhalation flow is above or equal to a first threshold. In other words, when the predicted operation parameter satisfies the first criterion an inhalation, exhalation, and/or operation has been determined to be successful. Formulated differently, when the predicted operation parameter satisfies the first criterion a medication intake by inhalation with the inhaler device has been determined to be successful.

In one or more example electronic devices, the processor is configured to determine whether one or more of the following satisfy the first criterion: an operation phase (such as a phase of an operation of the user when taking medication with the inhaler device), an inhalation feature, an inhaler device status, an activation feature, a container status, an inhalation flow feature, an amplitude feature, a time feature (such as duration feature), an inhalation volume feature, a flow feature, and a flow acceleration feature.

When it is determined that the predicted operation parameter does not satisfy the first criterion, the predicted operation parameter may be below the first threshold and/or outside a certain range indicative of the first criterion. When it is determined that the predicted operation parameter does not satisfy the first criterion, an inhalation, exhalation, and/or operation is determined to be unsuccessful. Formulated differently, when the predicted operation parameter does not satisfy the first criterion a medication intake by inhalation with the inhaler device may be determined to be unsuccessful.

In one or more example electronic devices, the first criterion comprises one or more of: a coordination criterion, an activation criterion, an inhaler status criterion, inhalation criterion, and a container status criterion. In one or more example electronic devices, the first criterion comprises one or more thresholds comprising one or more of: a first threshold associated with a coordination, a second threshold associated with an activation, a third threshold associated with an inhaler status, a fourth criterion associated with an inhalation (such as an inhalation flow), and a fifth threshold associated with a container status.

In other words, the processor may be configured to determine whether the predicted operation parameter is above, below, or equal a first threshold, a second threshold, a third threshold, a fourth threshold and/or a fifth threshold.

For example, the processor may be configured to determine whether the coordination parameter is above, below, or equal to the first threshold. In other words, the predicted operation parameter may satisfy the first criterion when the coordination parameter is above or equal to the first threshold.

For example, the processor may be configured to determine whether the activation parameter is above, below, or equal the second threshold. In other words, the predicted operation parameter may satisfy the first criterion when the activation parameter is above or equal to the second threshold.

For example, the processor may be configured to determine whether the inhaler status parameter is above, below, or equal the third threshold. In other words, the predicted operation parameter may satisfy the first criterion when the inhaler status parameter is above or equal to the third threshold.

For example, the processor may be configured to determine whether the inhalation parameter is above, below, or equal the fourth threshold. In other words, the predicted operation parameter may satisfy the first criterion when the inhalation parameter is above or equal to the fourth threshold. In other words, the predicted operation parameter may satisfy the first criterion when the predicted operation parameter is above or equal to the first inhalation flow threshold and/or the fourth threshold. For example, the predicted operation parameter may satisfy the first criterion when the inhalation flow is above or equal to the fourth threshold and/or an inhalation time is above, equal to, and/or within a range of the fourth threshold.

For example, the processor may be configured to determine whether the container status parameter is above, below, or equal the fifth threshold. In other words, the predicted operation parameter may satisfy the first criterion when the container status parameter is above or equal to the fifth threshold.

A coordination criterion may be seen as a criterion, such as timing criterion, related to a timing and/or a coordination of an activation of the inhaler device and an inhalation with the inhaler device. For example, when using a spray-type inhaler device, the coordination criterion may be satisfied when it is determined that the user has started inhaling first and then activated the inhaler device by actuating (e.g., by pressing) the container of the inhaler device to output a medication dose while continuing the inhalation. On the other hand, the coordination criterion may not be satisfied when it is determined that the user has unsuccessfully coordinated the activation of the inhaler device and the inhalation with the inhaler device.

An activation criterion may be seen as a criterion related to an activation of the inhaler device. For example, when using a spray-type inhaler device, the activation criterion may be satisfied when it is determined that the user has successfully activated the inhaler device by actuating (such as pressing) the container of the inhaler device and that a medication dose has been outputted. On the other hand, the activation criterion may not be satisfied when it is determined that the user has unsuccessfully activated the inhaler device, for example when the container has not been pressed enough and that an incomplete medication dose has been outputted.

An inhaler status criterion may be seen as a criterion related to an inhaler status of the inhaler device. For example, the inhaler status criterion may be satisfied when it is determined that the inhaler device is working correctly and/or does not need maintenance. On the other hand, the inhaler status criterion may not be satisfied when it is determined that the inhaler device is not working correctly and that it needs maintenance. For example, the inhaler status criterion may not be satisfied when it is determined that no sound was emitted by the inhaler device and/or that too much background noise was present in the audio signal.

In one or more example electronic devices, in accordance with the predicted operation parameter satisfying the first criterion, the processor is configured to determine a first recommendation. The first recommendation may be seen as a feedback to the user of the inhaler device regarding an operation (such as one or more operations) with the inhaler device. The first recommendation may be seen as a first evaluation. The first recommendation may be indicative of an inhalation, an exhalation, an activation, a coordination, and/or a medication intake that was successful or partly successful. The first recommendation may be indicative of an inhaler device status and/or a container status. The first recommendation may be seen as and/or comprise an advisory action that the user of the inhaler device should do. The first recommendation may for example comprise text (such as a message to the user) and/or phrases such as: “The medication intake was successful”, “Continue with your inhalation technique”, “Your inhalation was successful”, “Your inhalation was successful, but the medication container of the inhaler device is soon to be empty. Please verify the container status of the medication container of the inhaler device”, “The activation was successful”, “The coordination was successful”, “The inhaler device is working correctly”, and/or “The container is full or nearly full, you still have at least 10 medication doses and/or intakes left”. In one or more example electronic devices, when it is determined that the predicted operation parameter satisfies the first criterion, the processor may be configured to refrain from determining a first recommendation. Thereby the user would know that when no recommendation is outputted the operation was successful.

In one or more example electronic devices, the processor is configured to output, via the interface (such as the interface of the electronic device), the first recommendation. In other words, the processor may be configured to output the first recommendation in the form of a text (such as a message to a user) and/or a phrase, an evaluation score, image data (such as one or more images), a spectrogram, and/or a user interface as described herein.

In one or more example electronic devices, the first recommendation is comprised in the operation representation. In other words, the processor may be configured to include the first recommendation in the operation representation. For example, the processor may be configured to output an operation representation comprising the first recommendation.

In one or more example electronic devices, the first recommendation comprises one or more of: an inhaler device maintenance recommendation, a container recommendation, a coordination recommendation, an inhalation depth recommendation, an inhalation duration recommendation, an inhalation flow rate recommendation, and an inhalation preparation recommendation.

In one or more example electronic devices, the second recommendation comprises one or more of: an inhaler device maintenance recommendation, a container recommendation, a coordination recommendation, an inhalation depth recommendation, an inhalation duration recommendation, an inhalation flow rate recommendation, and an inhalation preparation recommendation.

An inhalation depth recommendation may be seen as a recommendation to the user of the inhaler device regarding the inhalation depth when performing the inhalation that the operation data is based on. The inhalation depth recommendation may indicate whether the inhalation depth of the user of the inhaler device when performing the inhalation that the operation data is based on was satisfying or not. For example, an inhalation depth recommendation may comprise “Your inhalation depth was satisfying”, “Your inhalation depth was not satisfying, please inhale deeper next time”, and/or, “Your inhalation depth was not satisfying, next time please repeat the inhalation operation (such as medication intake) and inhale deeper”.

An inhalation duration recommendation may be seen as a recommendation to the user of the inhaler device regarding the inhalation duration when performing the inhalation that the operation data is based on. The inhalation duration recommendation may indicate whether the inhalation duration of the user of the inhaler device when performing the inhalation that the operation data is based on was satisfying or not. For example, an inhalation duration recommendation may comprise “Your inhalation duration was satisfying”, “Your inhalation duration was not satisfying, please inhale for a longer time next time”, and/or “Your inhalation duration was not satisfying, next time please repeat the inhalation operation (such as medication intake) and inhale for a longer time”.

An inhalation flow recommendation may be seen as a recommendation to the user of the inhaler device regarding the inhalation flow when performing the inhalation that the operation data is based on. The inhalation flow recommendation may indicate whether the inhalation flow of the user of the inhaler device when performing the inhalation that the operation data is based on was satisfying or not. The inhalation flow recommendation may comprise a minimum inhalation flow recommendation, e.g., being indicative of a minimum inhalation flow that is recommended to the user for achieving a successful medication intake. For example, an inhalation flow recommendation may comprise “Your inhalation flow was satisfying”, “Your inhalation flow was not satisfying, please inhale with a higher flow next time”, and/or “Your inhalation flow was not satisfying, next time please repeat the inhalation operation (such as medication intake) and inhale with a higher flow”.

An inhalation preparation recommendation may be seen as a recommendation to the user of the inhaler device regarding the inhalation preparation when performing the inhalation that the operation data is based on. An inhalation preparation may for example comprise that the user starts inhaling before actuating and/or activating a medication container of the inhaler device. In other words, an inhalation preparation may comprise to coordinate an inhalation and actuation and/or activating of a medication container of the inhaler device before and/or when starting a medication intake operation. An inhalation preparation may for example comprise that the user clears his/her throat before starting a medication intake procedure (such as inhalation).

The inhalation preparation recommendation may indicate whether the inhalation preparation of the user of the inhaler device when performing the inhalation that the operation data is based on was satisfying or not. For example, an inhalation preparation recommendation may comprise “Your inhalation preparation was satisfying”, “Your inhalation preparation was not satisfying, please start inhaling before actuating and/or activating a medication container of the inhaler device next time”, and/or “Your inhalation preparation was not satisfying, next time please repeat the inhalation operation (such as medication intake) and start inhaling before actuating and/or activating a medication container of the inhaler device”.

An inhaler device maintenance recommendation may be seen as a recommendation to the user of the inhaler device regarding inhaler device maintenance after having performed the operation and/or inhalation that the operation data is based on. The inhaler device maintenance recommendation may indicate whether the inhaler device needs maintenance or not. The inhaler device maintenance recommendation may comprise a recommendation regarding a type of maintenance task to perform on the inhaler device. For example, an inhaler device maintenance recommendation may comprise “No or poor sound was emitted from your inhaler device, please examine your inhaler device and/or inhaler device container”, “Please clean your inhaler device”, “Your inhaler device does not seem to work properly anymore, please replace your inhaler device”, “A lot of background noise is present in the audio signal, please perform your operation in a more quiet environment”, and/or “No or poor sound has been obtained from your inhaler device, please move your electronic device and inhaler device closer to each other”.

A container recommendation may be seen as a recommendation to the user of the inhaler device regarding the container of the inhaler device, e.g., after having performed the operation and/or inhalation that the operation data is based on. The container recommendation may indicate whether the container of the inhaler device is operational or not. The container recommendation may comprise a recommendation regarding a type of actions to perform on the inhaler device and/or the container. For example, a container recommendation may comprise one or more recommendations such as: “Your inhalation was unsuccessful, because the medication container of the inhaler device was empty. Please change inhaler device or replace the medication container of the inhaler device”, “The container is soon to be empty, please be aware that the container will need to be replaced soon”, “The container is empty, please replace the container and/or the inhaler device”, “Please verify if the container is damaged”, “Please verify that you have the correct type of container mounted on the inhaler device”, “The activation of the container was successful”, and/or “The container of the inhaler device is not properly mounted on the inhaler device”.

A coordination recommendation may be seen as a recommendation to the user of the inhaler device regarding the coordination of one or more operations with the inhaler device, e.g., when performing an inhalation with the inhaler device. The coordination recommendation may indicate whether the coordination of the operation(s) with the inhaler device is successful or not. For example, a coordination recommendation may comprise some feedback regarding a coordination of an activation of the inhaler device (such as an actuation of the container of the inhaler device) and an inhalation with the inhaler device, such as a start or beginning of an inhalation. For example, a coordination recommendation may comprise one or more recommendations such as: “Your activation of the container and your inhalation was successfully coordinated”, “The coordination of the operation was successful”, and/or “The coordination was unsuccessful, next time please repeat the operation with a correct coordination”.

In one or more example electronic devices, in accordance with the predicted operation parameter not satisfying the first criterion, the processor is configured to determine a second recommendation. The second recommendation may be seen as a feedback to the user of the inhaler device regarding an operation (such as one or more operations) with the inhaler device. The second recommendation may be seen as a second evaluation. The second recommendation may be indicative of an inhalation, an exhalation, an activation, a coordination, and/or a medication intake that was unsuccessful or partly unsuccessful. The second recommendation may be indicative of an inhaler device status and/or a container status. The second recommendation may be seen as and/or comprise an advisory action that the user of the inhaler device should do. The second recommendation may for example comprise text (such as a message to the user) and/or phrases such as: “The medication intake was unsuccessful”, “Your inhalation technique needs to be improved”, “Your inhalation was unsuccessful”, “You exhaled too much when performing the medication intake”, “Your inhalation flow was satisfying, but the inhalation time was not sufficient”, “Your inhalation was unsuccessful, because the medication container of the inhaler device was empty. Please change inhaler device or replace the medication container of the inhaler device”, “The inhaler device needs to be replaced”, “The inhaler device needs to be cleaned”, “The activation was unsuccessful, next time please repeat the operation”, “The coordination was unsuccessful, next time please repeat the operation with a correct coordination”, “The container is soon to be empty, please be aware that the container will need to be replaced soon”, and/or “The container is empty, please replace the container and/or the inhaler device”.

A system for characterizing and/or monitoring an operation with an inhaler device is disclosed. The system comprises the inhaler device and an electronic device according to the present disclosure.

A method, for characterizing and/or monitoring an operation with an inhaler device is disclosed. The method comprises obtaining operation data, where the operation data is indicative of an audio signal representing an operation of the inhaler device. The method comprises determining, based on the operation data, e.g., using a predictor circuitry, a predicted operation parameter indicative of a prediction of an operation of the inhaler device. The method comprises determining, based on the predicted operation parameter, an operation representation. Optionally, the method comprises outputting, via the interface, the operation representation.

It is to be understood that a description of a feature in relation to the electronic device(s) is also applicable to the corresponding feature in the system(s), and/or the method(s) for characterizing and/or monitoring an operation of an inhaler device.

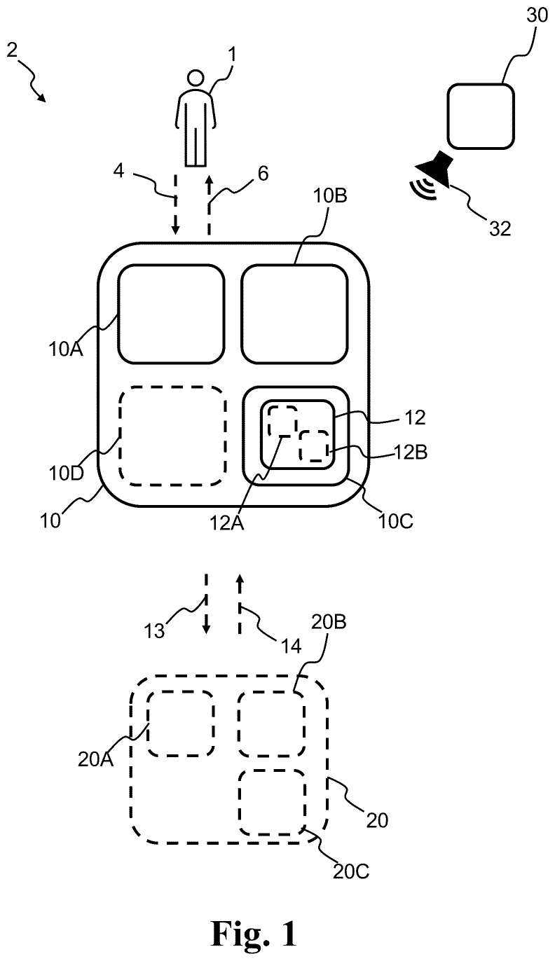

FIG. 1 schematically illustrates an exemplary system, such as a system 2 for characterizing and/or monitoring an operation with an inhaler device, according to the present disclosure. The system 2 comprises an inhaler device 30. The system 2 comprises an electronic device 10, such as the electronic device for characterizing and/or monitoring an operation with an inhaler device according to the present disclosure.

In other words, the electronic device 10 may be configured to characterize and/or monitor an operation of the inhaler device 30, such as an operation performed with the inhaler device 30. In other words, the electronic device 10 may be configured to characterize and/or monitor an operation performed by a user 1 when using the inhaler device 30. The electronic device 10 may be configured to characterize and/or monitor an operation performed with the inhaler device 30, such as an operation of the inhaler device 30 by the user 1. An operation may be seen as an operation of the inhaler device 30.

The electronic device 10 comprises a memory 10A, an interface 10B (such as one or more interfaces), and a processor 10C. Optionally, the system 2 comprises a server device 20. The server device 20 comprises a memory 20A, an interface 20B (such as one or more interfaces), and a processor 20C (such as one or more processors). The processor 10C comprises predictor circuitry 12 configured to operate according to a prediction model. In one or more example electronic devices and/or systems, the model as referred to herein may be stored on a non-transitory storage medium (for example, on the memory 10A of the electronic device 10, and/or on the memory 20A of the server device 20). In one or more example electronic devices and/or systems, the predictor circuitry 12 comprises a neural network module 12A configured to operate according to a neural network.

In one or more example electronic devices and/or systems, the predictor circuitry 12 comprises a neural network module 12A configured to operate according to a neural network.

In one or more example electronic devices and/or systems, the neural network is a deep neural network, such as a classification neural network configured to operate according to a classification model.

In one or more example electronic devices and/or systems, the predictor circuitry 12 comprises a regressor module 12B configured to operate according to a regression model.

In one or more example electronic devices and/or systems, the electronic device 10 is a user equipment device.

In one or more example electronic devices and/or systems, the electronic device 10 is a server device.

The electronic device 10 may comprise a user equipment device and/or a server device. The electronic device 10 may be configured to operate on a user equipment device and/or a server device. In other words, the electronic device 10 may be configured to act as a server device and/or a user equipment device. A user equipment device may for example be or comprise a mobile phone, such as a smartphone, a computer, such as a laptop computer or PC, or a tablet computer. In other words, the electronic device 10 may for example be a user device, such as a mobile phone or a computer, configured to perform a characterization and/or monitoring of an operation with the inhaler device 30. A server device (such as server device 20) may be configured on a cloud, such as a cloud network. Different operations configured to be performed by the electronic device 10 and/or the system 2 as disclosed herein may be performed at different devices, such as at the electronic device 10 and/or at the server device 20. In one or more example electronic devices and/or systems, when the electronic device 10 acts as a user equipment device, the system 2 may also comprise the server device 20.

The processor 10C is configured to obtain operation data (such as operation data and/or exhalation data), where the operation data is indicative of an audio signal (such as audio data) representing an operation with the inhaler device 30. In other words, the operation data may be based on an audio signal representing an operation with the inhaler device 30. For example, the operation data may be based on an audio signal from an operation, such as an operation and/or procedure, performed by the user 1 with the inhaler device 30. The user 1 may be a user of the inhaler device 30 taking a medication dose with the inhaler device 30. To obtain operation data may comprise that the processor is configured to determine, retrieve, generate, and/or receive the operation data. Optionally, the processor 10C may be configured to obtain 14 the operation data from the server device 20, e.g., via a network, such as a global network as the internet, using the interface 10B. The operation data may be seen as an audio recording of an operation, such as an operation, performed by the user 1 with the inhaler device 30. In other words, the operation data may be seen as an audio recording of a sound sequence of an operation performed by the user 1 with the inhaler device 30, such as a sound output 32 produced by an inhalation, an exhalation, an amplifier of the inhaler device 30, and/or a container of the inhaler device, 30.

The processor 10C is configured to determine, based on the operation data, using the predictor circuitry 12, a predicted operation parameter indicative of a prediction of an operation of the inhaler device 30.

In one or more example electronic devices and/or systems, the audio signal has a flow dependent sound frequency profile. In other words, the inhaler device 30 may be configured to generate a flow-dependent sound frequency profile that the audio signal is based on. For example, the inhaler device 30, such as acoustic inhaler device, may be configured to generate a sound output 32 (such as an audio signal) having a flow dependent sound frequency profile. The inhaler device 30 may comprise an acoustic amplifier, such as a whistle, configured to generate a sound output 32 (such as an audio signal) comprising a flow-dependent sound frequency profile. For example, the inhaler device 30 comprises a hole tone whistle configured to generate an audio signal comprising a flow-dependent sound frequency profile.

In one or more example electronic devices and/or systems, the electronic device 10 comprises one or more microphones 10D for obtaining (such as generating) the audio signal. For example, the electronic device 10 may comprise a mobile phone comprising one or more microphones 10D for obtaining the audio signal. In other words, the electronic device comprises one or more microphones 10D configured to generate and/or provide the audio signal based on a sound output 32 generated by the inhaler device 30, such as the acoustic inhaler device.

In one or more example electronic devices and/or systems, the obtaining of the operation data comprises to obtain, based on the audio signal, sound data of the audio signal, and wherein the determination of the predicted operation parameter is based on the sound data. In one or more example electronic devices and/or systems, the processor 10C is configured to obtain, based on the audio signal, sound data of the audio signal. In one or more example electronic devices and/or systems, the processor 10C is configured to determine the predicted operation parameter based on the sound data. In other words, the processor 10C may be configured to extract one or more audio features from the audio signal, where the sound data comprises the one or more audio features. It may be appreciated that the prediction model may be configured to extract one or more audio features from the audio signal to obtain (such as provide) the sound data. The sound data may comprise one or more audio features, such as a sound frequency feature and/or an amplitude feature.

In one or more example electronic devices and/or systems, the sound data comprises one or more of: a frequency signature, an amplitude signature, and a duration signature. For example, the predictor circuitry 12 may be configured to classify, identify, and/or detect (such as using the prediction model, e.g., a classification model) a signature, such as a frequency signature, an amplitude signature, and/or a duration signature. A signature, such as a frequency signature, an amplitude signature, and/or a duration signature may for example be classified, identified, and/or detected for the same user and/or across several users. It may for example be possible to classify, identify, and/or detect a condition of a user based on the sound data. In other words, a signature may be indicative of a condition of a user, such as lung condition and/or a respiratory condition. Furthermore, it may be possible to classify, identify, and/or detect an anomaly, such as an anomaly of the inhaler device, an anomaly of the use of the inhaler device, and/or an anomaly of a microphone of the electronic device. A signature may be seen as a classification, an identification and/or a detection of a pattern.

In one or more example electronic devices and/or systems, the obtaining of the operation data comprises to perform pre-processing of the audio signal. In one or more example electronic devices and/or systems, the processor 10C may be configured to perform pre-processing of the audio signal to obtain, such as determine, the operation data. In other words, the processor 10C may comprise a pre-processing module (not shown) configured to perform pre-processing of the audio signal. For example, the obtaining of the operation data may comprise to apply one or more filters to the audio signal and/or to transform the audio signal. In one or more example electronic devices and/or systems, the obtaining of the operation data comprises to apply one or more filters to the audio signal. In one or more example electronic devices and/or systems, the processor 10C may be configured to apply one or more filters to the audio signal to obtain, such as determine, the operation data. In other words, the processor 10C comprises a pre-processing module (not shown) configured to apply one or more filters to the audio signal. To apply one or more filters may comprise to apply a low-pass filter and/or a high-pass filter to the audio signal. By applying one or more filters to the audio signal the signal to noise ratio (SNR) may be enhanced. In one or more example electronic devices and/or systems, the prediction circuitry 12 is configured to perform pre-processing of the audio signal by using the prediction model on the audio signal. In other words, the prediction model may be configured to (such as used to) extract the audio signal representing an operation with an inhaler device from an audio signal (such as audio data) by applying one or more filters.

In one or more example electronic devices and/or systems, the obtaining of the operation data comprises to identify a background noise (such as background sounds) from the audio signal. In one or more example electronic devices and/or systems, the processor 10C may be configured to identify a background noise from the audio signal. In one or more example electronic devices and/or systems, the prediction circuitry 12 is configured to identify a background noise by using the prediction model on the audio signal. For example, the prediction model may be pre-trained based on a plurality of background noises in order to be able to identify one or more background noises from the audio signal. In other words, the prediction model may be trained to extract the audio signal representing an operation with the inhaler device 30 from an audio signal (such as audio data) comprising one or more background noises (such as background sounds).

In one or more example electronic devices and/or systems, the obtaining of operation data comprises to split (such as cut) the audio signal (such as audio data) into a plurality of audio samples. In one or more example electronic devices and/or systems, the processor 10C is configured to split the audio signal into a plurality of audio samples. For example, the processor 10C may comprise an audio processing module (not shown) configured to process the audio signal, such as configured to split the audio signal into a plurality of audio samples. In other words, by splitting the audio signal (such as audio data) into a plurality of audio samples it may be possible to divide the audio signal into different phases of the operation.

In one or more example electronic devices and/or systems, the determination of the predicted operation parameter is based on one or more audio samples of the plurality of audio samples. In one or more example electronic devices and/or systems, the processor 10C is configured to determine the predicted operation parameter based on one or more audio samples of the plurality of audio samples. In other words, the predictor circuitry 12 may be configured to provide one or more audio samples of the plurality of audio samples as an input to the prediction model. In other words, the processor 10C (such as the predictor circuitry 12) may be configured to provide the plurality of the audio samples as an input to the prediction model and keep the structure of the audio signal, such as keep the chronology of the audio samples.

In one or more example electronic devices and/or systems, the obtaining of operation data comprises to shuffle the plurality of audio samples. In one or more example electronic devices and/or systems, the processor 10C is configured to shuffle the plurality of audio samples. In one or more example electronic devices and/or systems, the determination of the predicted operation parameter is based on the shuffled audio samples.

In one or more example electronic devices and/or systems, the processor 10C is configured to determine the predicted operation parameter based on the shuffled audio samples. In other words, the predictor circuitry 12 may be configured to provide one or more of the shuffled audio samples of the plurality of audio samples as an input to the prediction model. In other words, the processor 10C (such as the predictor circuitry 12) may be configured to provide the plurality of shuffled audio samples as an input to the prediction model. By providing the plurality of shuffled audio samples as an input to the prediction model the prediction model may be trained to determine the predicted operation parameter without having the structure and/or chronology of the audio samples. This may provide a more robust prediction model to determine predicted operation parameters. Further, by splitting (such as cutting) and/or shuffling the audio signal, more audio data is provided, for example to train the prediction model, expand the audio data library, and/or determine predicted operation parameters on smaller audio samples. It may be appreciated that it may be possible to determine a plurality of predicted operation parameters based on the same inhalation (such as inhalation operation). It may be appreciated that the trained of the prediction model may be performed on the server device 20 or when the electronic device 10 acts as a server device on the electronic device 10.

In one or more example electronic devices and/or systems, the obtaining of operation data comprises to transform the audio signal to a spectrogram. In one or more example electronic devices and/or systems, the processor 10C is configured to transform the audio signal to a spectrogram.

In one or more example electronic devices and/or systems, the determination of the predicted operation parameter is based on the spectrogram. In one or more example electronic devices and/or systems, the processor 10C is configured to determine the predicted operation parameter based on the spectrogram.

In one or more example electronic devices and/or systems, the obtaining of the operation data comprises to obtain image data, such as based on the audio signal. In one or more example electronic devices and/or systems, the processor 10C is configured to obtain image data. In one or more example electronic devices and/or systems, the determination of the predicted operation parameter is based on the image data. In one or more example electronic devices and/or systems, the processor 10C is configured to determine the predicted operation parameter based on the image data. In other words, the processor 10C is configured to use the image data as input to the prediction model. In one or more example electronic devices and/or systems, the processor 10C is configured to determine one or more image features based on the image data. The one or more image features may be indicative of one or more of: an operation phase (such as a phase of an operation of the user when taking medication with the inhaler device), an inhalation flow, an amplitude, time (such as duration), an inhalation volume, and an inhalation flow acceleration.

In one or more example electronic devices and/or systems, the processor 10C is configured to determine the predicted operation parameter based on the one or more features.

The processor 10C is configured to determine, based on the operation data, using the predictor circuitry 12, a predicted operation parameter indicative of a prediction of an operation of the inhaler device 30. An operation as disclosed herein may be seen as one or more operations, such as comprising a first operation, a second operation, and/or a third operation. An operation as disclosed herein may comprise a series of operations. In other words, the processor 10C is configured to determine, based on the operation data, using the prediction model, a predicted operation parameter indicative of a prediction of an operation of the inhaler device 30. In one or more example electronic devices and/or systems, the processor 10C is configured to determine, based on the operation data, using the predictor circuitry 12, a predicted operation parameter indicative of an inhalation flow and/or an exhalation flow. Determining a predicted operation parameter indicative of a prediction of an operation may comprise extracting one or more operation features from the audio signal and/or the operation data. In other words, determining a predicted operation parameter indicative of a prediction of an operation may be based on one or more features extracted from the audio signal and/or the operation data. In one or more example electronic devices and/or systems, the processor 10C is configured to determine the predicted operation parameter based on one or more features extracted from the audio signal and/or the operation data. For example, when the audio signal has a flow dependent sound frequency, the processor 10C may be configured to extract a sound frequency from the audio signal, and to determine an inhalation flow and/or an exhalation flow based on the extracted sound frequency.

In one or more example electronic devices and/or systems, the determination of the predicted operation parameter comprises to determine one or more of: an inhalation parameter, an inhaler device status parameter, an activation parameter, a coordination parameter, and a container status parameter. In other words, the electronic device 10, such as the processor 10C, is configured to determine, e.g., using the predictor circuitry 12, one or more of: an inhalation parameter, an inhaler device status parameter, an activation parameter, a coordination parameter, and a container status parameter. An inhalation parameter may be seen as a parameter indicative of an inhalation (such as an inhalation operation) performed with the inhaler device, such as an inhalation performed by a user of the inhaler device. An inhalation parameter may be seen as an inhalation characteristic. An inhalation parameter may comprise one or more inhalation flows, such as an inhalation flow over time.

In one or more example electronic devices and/or systems, the determination of the container status parameter comprises to determine whether a container is present in the inhaler device 30. In other words, the electronic device 10 is configured to determine whether a container is present in the inhaler device 30. To determine whether a container is present may comprise to determine whether a container (such as canister and/or a capsule) is correctly mounted in the inhaler device 30. This may be particularly advantageous when using inhaler devices with add-on containers, where the user may have to mount the container on the inhaler device 30. By determining whether a container is present in the inhaler device 30, it may be possible to determine whether a container was present when the user performed an operation (such as an inhalation) with the inhaler device 30. To determine whether a container is present may comprise to determine which type of container is present in the inhaler device 30. For example, when using a capsule-type inhaler device, the capsule will rotate while performing an inhalation, making a different sound than when using a canister-type inhaler device.

In accordance with the determination that a container is present in the inhaler device 30, the electronic device 10 is configured to determine a recommendation, such as a first recommendation as disclosed herein. The electronic device 10 may for example, in response to the determination that a container was present, be configured to determine one or more recommendations such as: “A container is mounted on your inhaler device, you should be able to perform a successful inhalation with your inhaler device”, and/or “It was established that a container was mounted on your inhaler device while performing an operation”. Alternatively or additionally, the electronic device may be configured to refrain from determining a recommendation when it is determined that a container is present in the inhaler device. The determination of whether a container is present in the inhaler device 30 or not may be used by the electronic device 10, such as the processor 10C, to determine whether an operation was successful or not.

In one or more example electronic devices and/or systems, in accordance with the determination that a container is not present in the inhaler device 30, the electronic device 10 is configured to determine a recommendation, such as a second recommendation and/or a container recommendation as disclosed herein. The electronic device 10 may for example, in response to the determination that no container was present, be configured to determine one or more recommendations such as: “There is no container mounted on your inhaler device, please mount a container on the inhaler device”, and/or “It was established that no container was mounted on your inhaler device, please verify that a container is mounted and/or that the container is correctly mounted”.

In one or more example electronic devices and/or systems, the determination of the container status parameter comprises to determine a content of a container of the inhaler device 30. In other words, the electronic device 10 is configured to determine a content of a container in or of the inhaler device 30. To determine a content of a container may comprise to determine whether a container (such as canister and/or a capsule) is full, half-full, empty, and/or monitor the emptying of the container during an operation. To determine a content of a container may comprise to determine content of medication left in the container. In one or more example electronic devices and/or systems, the user may provide an input indicating a type of container of the inhaler device 30. The determination of a content of a container may be used to determine how a medication dose was outputted during the inhalation, e.g., if a container (such as capsule) emptied completely or not during the inhalation.

In one or more example electronic devices and/or systems, the processor 10C is configured to determine, such as using the predictor circuitry 12, based on the predicted operation parameter, one or more of: a duration of the operation, a coordination of the operation, an inhalation volume, an average inhalation flow, a maximum inhalation flow, a minimum inhalation flow, median inhalation flow, an inhalation flow acceleration, an inhalation pattern, and one or more exhalation parameters. In other words, the predicted operation parameter may comprise one or more of: a duration of the operation, a coordination of the operation(s), an inhalation volume, an average inhalation flow, a maximum inhalation flow, a minimum inhalation flow, median inhalation flow, an inhalation flow acceleration, and one or more exhalation parameters. In other words, an output of the prediction model may comprise one or more of: a duration of the operation, a coordination of the operation, an inhalation volume, an average inhalation flow, a maximum inhalation flow, a minimum inhalation flow, median inhalation flow, an inhalation flow acceleration, and one or more exhalation parameters. In one or more example electronic devices and/or systems, the processor 10C is configured to determine, such as using the predictor circuitry 12, based on the operation data and/or the audio signal, one or more of: a duration of the operation, a coordination of the operation, an inhalation volume, an average inhalation flow, a maximum inhalation flow, a minimum inhalation flow, median inhalation flow, an inhalation flow acceleration, and one or more exhalation parameters.

The processor 10C is configured to determine, based on the predicted operation parameter, an operation representation. The operation representation may be seen as a representation indicative of all or part of the operation performed by a user with the inhaler device 30. In other words, the operation representation may be seen as an inhaler device operation representation. The operation representation may be seen as and/or comprise an evaluation of an operation with the inhaler device 30, such as an operation evaluation and/or an inhalation evaluation. In other words, the operation representation may be seen as and/or comprise an evaluation of an operation of a user when taking a medication with the inhaler device 30.

In one or more example electronic devices and/or systems, the operation representation comprises a representation of the predicted operation parameter. For example, the operation representation may comprise a representation of an inhalation flow with the inhaler device, such as a graph or a plot of the inhalation flow over time, a spectrogram and/or the image data as described herein. The operation representation may comprise one or more of: a representation of a duration of inhalation, a representation of an inhalation volume, a representation of an inhalation flow profile, a representation of an average inhalation flow, a representation of a maximum inhalation flow, a representation of a minimum inhalation flow, a representation of a median inhalation flow, a representation of an inhalation flow acceleration, and a representation of one or more exhalation parameters. The operation representation may be indicative of the whole operation with the inhaler device, and/or part of the operation with the inhaler device 30, such as a phase of the operation with the inhaler device 30.

In one or more example electronic devices and/or systems, the operation representation is indicative of an evaluation of the inhaler device 30. In other words, the processor 10C may be configured to determine a state of the inhaler device 30 based on the operation data and/or the audio signal.

An advantage of having an operation representation, e.g. after an inhalation, an exhalation, and/or an operation with the inhaler device 30 has been performed, may be that the user 1 of the electronic device 10 and/or the inhaler device 30 may see or be informed right after the inhalation, such as operation, about his/her performance or the outcome of the inhalation in relation to the predicted operation parameter. Therefore, when an inhalation was unsuccessful the user may be aware of it and may be able to repeat the inhalation and/or operation and/or improve his/her performance for the next inhalation and/or operation. Furthermore, the user 1 of the electronic device 10 and/or the inhaler device 30 may get a better feedback on his/her performance or on the outcome of the inhalation and/or operation. The operation representation may provide a gamification of the users' performances. The operation representation may for example increase the intelligibility of the feedback to the user 1, e.g. by being able to visualize an inhalation and/or operation with the inhaler device 30, and further to improve his/her inhalation technique by being able to visualize an improvement of performances.

Optionally, the processor 10C is configured to output, via the interface 10B, the operation representation. Optionally, the processor 10C is configured to output 13, via the interface 10B, the operation representation to the server device 20. Optionally, the processor 10C is configured to output 6, via the interface 10B, the operation representation to the user 1.

In one or more example electronic devices and/or systems, the operation representation is indicative of a performance of the operation of the inhaler device 30. For example, the operation representation may be indicative of a low performance when an inhalation was too low (e.g., based on the sound volume), an inhalation was too short, an inhalation was performed with an empty container, and/or that a coordination of operation(s) was faulty and/or wrong.

In one or more example electronic devices and/or systems, the operation representation comprises an operation score indicative of a performance of a user 1 when performing an operation with the inhaler device 30, such as a performance of a medication intake. The operation representation may indicate whether an operation, an activation, a coordination of operation(s), an inhalation, an exhalation, and/or a medication intake was successful or not. The operation representation may provide information to the user 1 regarding an inhaler device status, such as information regarding maintenance of the inhaler device 30. The operation representation may provide information to the user 1 regarding a container and/or a container status of the inhaler device 30, such as information regarding a presence of a container and/or regarding a content of a container. For example, the score may be indicative of a successful inhalation, exhalation, and/or medication intake when the predicted operation parameter satisfies a criterion. For example, the processor 10C may be configured to determine whether the score satisfies a criterion (such as the first criterion). When the score is above or equal to a threshold (such as the first threshold), it may be determined that the score satisfies the criterion (such as first criterion).

In one or more example electronic devices and/or systems, the operation representation comprises a representation of the predicted operation parameter. For example, the operation representation may comprise a representation of an inhalation flow with the inhaler device 30, such as a graph or a plot of the inhalation flow over time, a spectrogram and/or the image data as described herein. The operation representation may comprise one or more of: a representation of an inhalation parameter, a representation of an inhaler device status, a representation of an activation, a representation of a container status, a representation of a duration of inhalation, a representation of an inhalation volume, a representation of an inhalation flow profile, a representation of an average inhalation flow, a representation of a maximum inhalation flow, a representation of a minimum inhalation flow, a representation of a median inhalation flow, a representation of an inhalation flow acceleration, and a representation of one or more exhalation parameters. The operation representation may be indicative of the whole operation with the inhaler device 30, and/or part of the operation with the inhaler device 30, such as a phase of the operation with the inhaler device 30.

In one or more example electronic devices and/or systems, the operation representation is indicative of an evaluation of the inhaler device 30. In other words, the processor 10C may be configured to determine a state of the inhaler device 30 based on the operation data and/or the audio signal.

Optionally, the processor 10C is configured to output 6, via the interface 10B, the operation representation.

In one or more example electronic devices and/or systems, outputting 6 the operation representation may comprise outputting 6, via the interface 10B of the electronic device 10, the operation representation. Outputting 6 the operation representation may comprise displaying a user interface indicative of the operation representation. In one or more exemplary electronic devices, outputting 6 the operation representation may comprise outputting 6, via the interface 10B of the electronic device 10, a first operation representation, a second operation representation, a third operation representation, etc.

Outputting 6 the operation representation may comprise displaying a user interface indicative of the operation representation.

In one or more example electronic devices and/or systems, the processor 10C is configured to determine whether the predicted operation parameter satisfies a first criterion. In other words, the processor 10C may be configured to determine whether the predicted operation parameter is above, below, or equal to an inhalation flow threshold and/or is within a certain range indicative of the first criterion. In other words, the predicted operation parameter may satisfy the first criterion when the predicted operation parameter is above or equal to a first threshold. For example, the predicted operation parameter may satisfy the first criterion when the inhalation flow is above or equal to a first threshold. In other words, when the predicted operation parameter satisfies the first criterion an inhalation, exhalation, and/or operation has been determined to be successful. Formulated differently, when the predicted operation parameter satisfies the first criterion a medication intake by inhalation with the inhaler device has been determined to be successful.

In one or more example electronic devices and/or systems, the first criterion comprises one or more of: a coordination criterion, an activation criterion, an inhaler status criterion, inhalation criterion, and a container status criterion. In one or more example electronic devices and/or systems, the first criterion comprises one or more thresholds comprising one or more of: a first threshold associated with a coordination, a second threshold associated with an activation, a third threshold associated with an inhaler status, a fourth criterion associated with an inhalation (such as an inhalation flow), and a fifth threshold associated with a container status.

In other words, the processor 10C may be configured to determine whether the predicted operation parameter is above, below, or equal a first threshold, a second threshold, a third threshold, a fourth threshold and/or a fifth threshold.

In one or more example electronic devices and/or systems, in accordance with the predicted operation parameter satisfying the first criterion, the processor 10C is configured to determine a first recommendation. The first recommendation may be seen as a feedback to the user of the inhaler device regarding an operation (such as one or more operations) with the inhaler device. The first recommendation may be seen as a first evaluation. The first recommendation may be indicative of an inhalation, an exhalation, an activation, a coordination, and/or a medication intake that was successful or partly successful. The first recommendation may be indicative of an inhaler device status and/or a container status. The first recommendation may be seen as and/or comprise an advisory action that the user of the inhaler device should do. The first recommendation may for example comprise text (such as a message to the user) and/or phrases such as: “The medication intake was successful”, “Continue with your inhalation technique”, “Your inhalation was successful”, “Your inhalation was successful, but the medication container of the inhaler device is soon to be empty. Please verify the container status of the medication container of the inhaler device”, “The activation was successful”, “The coordination was successful”, “The inhaler device is working correctly”, and/or “The container is full or nearly full, you still have at least 10 medication doses and/or intakes left”. In one or more example electronic devices, when it is determined that the predicted operation parameter satisfies the first criterion, the processor may be configured to refrain from determining a first recommendation. Thereby the user would know that when no recommendation is outputted the operation was successful.

In one or more example electronic devices and/or systems, the processor 10C is configured to output, via the interface (such as the interface 10B of the electronic device 10), the first recommendation. In other words, the processor 10C may be configured to output the first recommendation in the form of a text (such as a message to a user) and/or a phrase, an evaluation score, image data (such as one or more images), a spectrogram, and/or a user interface as described herein. In one or more example electronic devices and/or systems, the processor 10C is configured to output, via the interface 10B, the first recommendation. Optionally, the processor 10C is configured to output 13, via the interface 10B, the first recommendation to the server device 20. Optionally, the processor 10C is configured to output 6, via the interface 10B, the first recommendation to the user 1.

In one or more example electronic devices and/or systems, the first recommendation is comprised in the operation representation. In other words, the processor 10C may be configured to include the first recommendation in the operation representation. For example, the processor 10C may be configured to output 6, e.g., via the interface 10B, an operation representation comprising the first recommendation.

In one or more example electronic devices and/or systems, the first recommendation comprises one or more of: an inhaler device maintenance recommendation, a container recommendation, a coordination recommendation, an inhalation depth recommendation, an inhalation duration recommendation, an inhalation flow rate recommendation, and an inhalation preparation recommendation.

In one or more example electronic devices and/or systems, the second recommendation comprises one or more of: an inhaler device maintenance recommendation, a container recommendation, a coordination recommendation, an inhalation depth recommendation, an inhalation duration recommendation, an inhalation flow rate recommendation, and an inhalation preparation recommendation.

In one or more example electronic devices and/or systems, in accordance with the predicted operation parameter not satisfying the first criterion, the processor 10C is configured to determine a second recommendation. The second recommendation may be seen as a feedback to the user 1 of the inhaler device 30 regarding an operation (such as one or more operations) with the inhaler device 30. The second recommendation may be seen as a second evaluation. The second recommendation may be indicative of an inhalation, an exhalation, an activation, a coordination, and/or a medication intake that was unsuccessful or partly unsuccessful. The second recommendation may be indicative of an inhaler device status and/or a container status. The second recommendation may be seen as and/or comprise an advisory action that the user 1 of the inhaler device 30 should do. The second recommendation may for example comprise text (such as a message to the user) and/or phrases such as: “The medication intake was unsuccessful”, “Your inhalation technique needs to be improved”, “Your inhalation was unsuccessful”, “You exhaled too much when performing the medication intake”, “Your inhalation flow was satisfying, but the inhalation time was not sufficient”, “Your inhalation was unsuccessful, because the medication container of the inhaler device was empty. Please change inhaler device or replace the medication container of the inhaler device”, “The inhaler device needs to be replaced”, “The inhaler device needs to be cleaned”, “The activation was unsuccessful, next time please repeat the operation”, “The coordination was unsuccessful, next time please repeat the operation with a correct coordination”, “The container is soon to be empty, please be aware that the container will need to be replaced soon”, and/or “The container is empty, please replace the container and/or the inhaler device”.

Optionally, the electronic device 10 may be configured to output 6 (such as user output), such as via a display interface of the imaging device 10 and/or a separate display interface of a separate electronic device, a user interface comprising a plurality of user interface objects to a user 1 of the electronic device. The electronic device 10, such as the processor 10C, may be configured to output 6, such as via the interface 10B, the operation representation to the user 1. In other words, the electronic device 10, such as the processor 10C, may be configured to output 6, such as display, a user interface comprising the operation representation to the user 1.

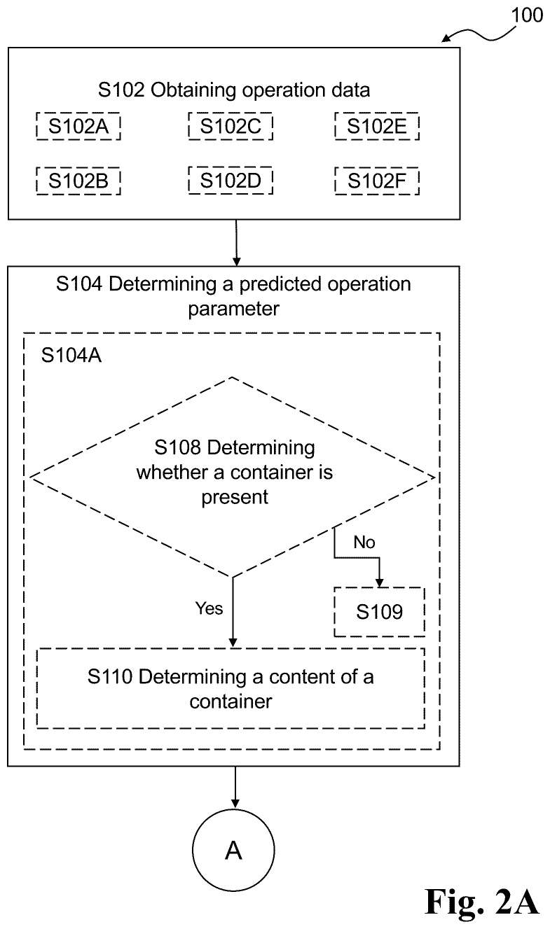

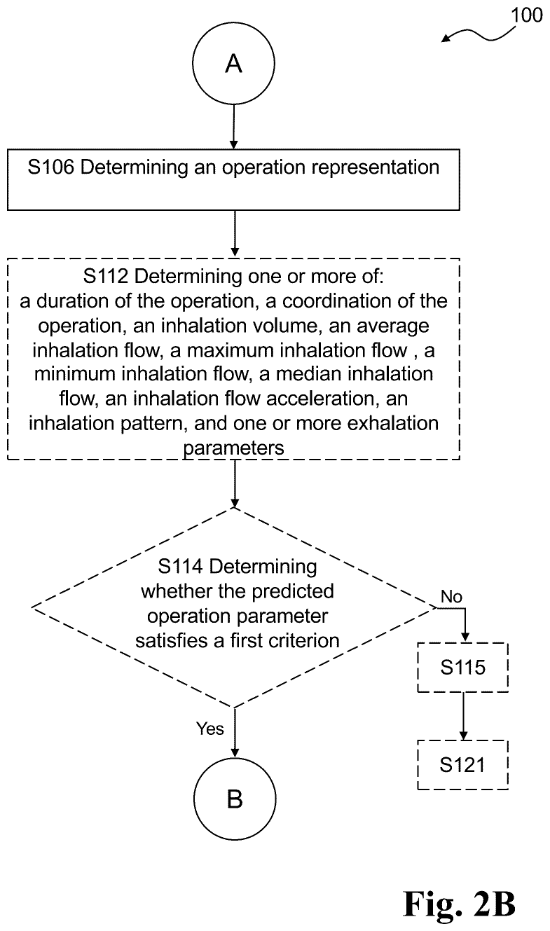

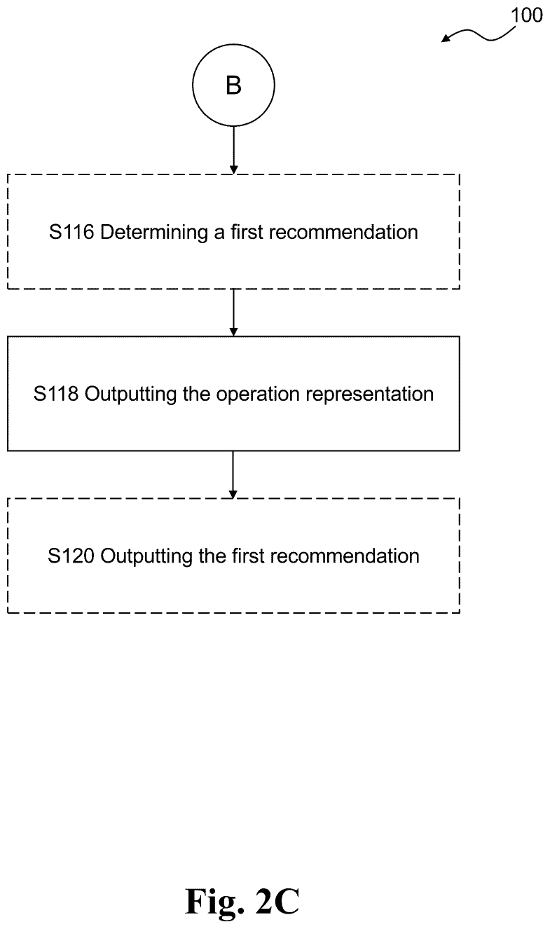

Optionally, the user 1 may provide an input 4 (such as user input), such as via the interface 10B, to the electronic device 10. The determination of one or more of the operation data, the predicted operation parameter, and the operation representation may be based on the input 4 from the user. The user 1 may for example provide an inhaler device type and/or user information about himself. The electronic device 10 may be configured to perform any of the methods disclosed in FIGS. 2A, 2B.

The processor 10C is optionally configured to perform any of the operations disclosed in FIGS. 2A-2B (such as any one or more of S102A, S102B, S102C, S102D, S102E, S102F, S104A, S106, S108, S109, S110, S112, S114, S115, S116, S118, S120, S121). The operations of the electronic device 10 may be embodied in the form of executable logic routines (for example, lines of code, software programs, etc.) that are stored on a non-transitory computer readable medium (for example, memory 10A) and are executed by the processor 10C).

Furthermore, the operations of the electronic device 10 may be considered a method that the electronic device 10 is configured to carry out. Also, while the described functions and operations may be implemented in software, such functionality may as well be carried out via dedicated hardware or firmware, or some combination of hardware, firmware and/or software.

Memory 10A may be one or more of a buffer, a flash memory, a hard drive, a removable media, a volatile memory, a non-volatile memory, a random access memory (RAM), or other suitable device. In a typical arrangement, memory 10A may include a non-volatile memory for long term data storage and a volatile memory that functions as system memory for the processor 10C. The memory 10A may exchange data with the processor 10C over a data bus. Control lines and an address bus between the memory 10B and the processor 10C also may be present (not shown in FIG. 1). The memory 10A is considered a non-transitory computer readable medium.

The memory 10A may be configured to store information such as operation data, sound data, audio data, image data, predicted operation parameter(s), operation representation(s), recommendation(s), and/or prediction model(s) as disclosed herein in a part of the memory.

The server device 20, such as the processor 20C, may be configured to perform any of the operations performed by the electronic device 10, such as the processor 10C, as described herein.

In other words, the description related to the processor 10C may apply to the description of the processor 20C. For example, when the electronic device 10 acts as a server device, the electronic device 10 and the server device 20 may be considered as one device.

FIGS. 2A and 2B show a flow diagram of an exemplary method, such as a method 100. A method 100, for characterizing and/or monitoring an operation of an inhaler device is disclosed. The method 100, may be performed by an electronic device and/or a system as disclosed herein (such as electronic device 10 and/or system 2).

The method 100 comprises obtaining S102 operation data, where the operation data is indicative of an audio signal representing an operation of the inhaler device.

The method 100 comprises determining S104, based on the operation data, a predicted operation parameter indicative of an operation of the inhaler device.

The method 100 comprises determining S106, based on the predicted operation parameter, an operation representation. The method 100 comprises outputting S118, the operation representation.

In one or more example methods, the determination S104 of the predicted operation parameter comprises determining S104A one or more of: an inhalation parameter, an inhaler device status parameter, an activation parameter, and a container status parameter.

In one or more example methods, the determination of the container status parameter comprises determining S108 whether a container is present in the inhaler device.

In accordance with the determination that a container is present in the inhaler device, the method may comprise determining a recommendation, such as a first recommendation as disclosed herein. Alternatively or additionally, the method may comprise refraining from determining a recommendation when it is determined that a container is present in the inhaler device. The determination of whether a container is present in the inhaler device or not may be used for determining whether an operation was successful or not.

In one or more example methods, in accordance with the determination that a container is not present in the inhaler device, the method may comprise determining S109 a recommendation, such as a second recommendation and/or a container recommendation as disclosed herein.

In one or more example methods, the determination of the container status parameter comprises determining S110 a content of a container of the inhaler device.

In one or more example methods, the method 100 comprises determining S112, based on the predicted operation parameter, one or more of: a duration of the operation, a coordination of the operation, an inhalation volume, an average inhalation flow, a maximum inhalation flow, a minimum inhalation flow, a median inhalation flow, an inhalation flow acceleration, an inhalation pattern, and one or more exhalation parameters.

In one or more example methods, the method 100 comprises determining S114 whether the predicted operation parameter satisfies a first criterion.

In one or more example methods, the method 100 comprises in accordance with the predicted operation parameter satisfying the first criterion, determining S116 a first recommendation. In one or more example methods, the method 100 comprises outputting S120, the first recommendation.

In one or more example methods, the method 100 comprises in accordance with the predicted operation parameter not satisfying the first criterion, determining S115 a second recommendation.

In one or more example methods, the method 100 comprises outputting S121, the second recommendation.

In one or more example methods, the obtaining S102 of the operation data comprises obtaining S102A, based on the audio signal, sound data of the audio signal. In one or more example methods, the determination S104 of the predicted operation parameter is based on the sound data.

In one or more example methods, the obtaining S102 of the operation data comprises performing S102B pre-processing of the audio signal.

In one or more example methods, the obtaining S102 of the operation data comprises identifying S102C a background noise from the audio signal.

In one or more example methods, the obtaining S102 of the operation data comprises splitting S102D the audio signal into a plurality of audio samples. In one or more example methods, the determination S104 of the predicted operation parameter is based on one or more audio samples of the plurality of audio samples.

In one or more example methods, the obtaining S102 of the operation data comprises shuffling S102E the plurality of audio samples. In one or more example methods, the determination S104 of the predicted operation parameter is based on the shuffled audio samples.

In one or more example methods, the obtaining S102 of the operation data comprises transforming S102F the audio signal to a spectrogram. In one or more example methods, the determination S104 of the predicted operation parameter is based on the spectrogram.

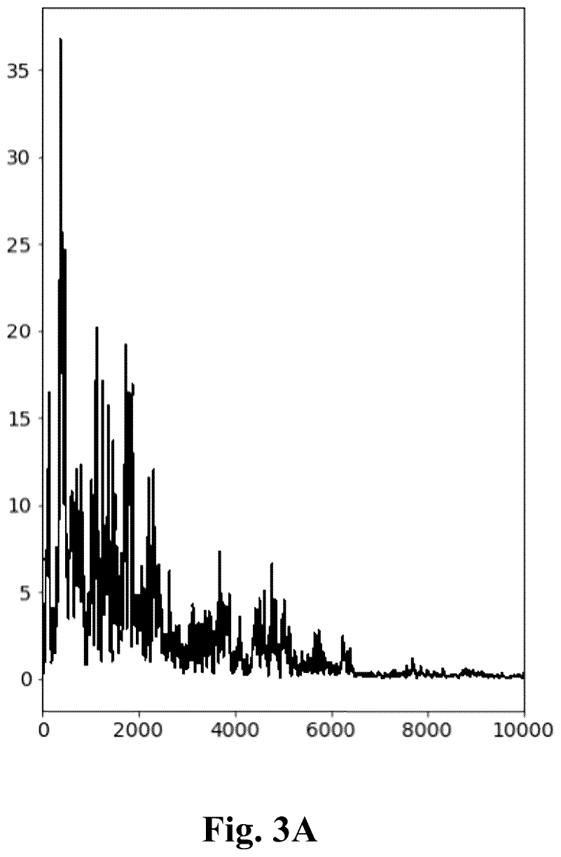

FIGS. 3A-3B show an example scenario of characterization and/or monitoring of an operation of an inhaler device where the technique disclosed herein is applied. In other words, FIGS. 3A-3B show an example scenario where an electronic device according to the present disclosure has been used to characterize and/or monitor an operation of an inhaler device.

FIGS. 3A-3B show a visualization (such as representation) of operation data indicative of an audio signal representing an operation of the inhaler device. The operation data is in FIG. 3A visualized (such as represented) in the form of a plot of an inhalation sound profile. The operation data has been obtained by the processor (such as processor 10C of FIG. 1).

FIGS. 3A-3B show an example representation of operation data indicative of an audio signal representing an operation with an inhaler device as disclosed herein. FIG. 3A is a graph representing frequency (such as sound frequency) in Hz on the X-axis and magnitude (such as energy) on the Y-axis. The magnitude is a relative difference in magnitude and is therefore unitless. The graph of FIG. 3A represents the magnitude with respect to frequency for an inhalation performed with an inhaler device of a metered dose inhaler type without acoustic amplifier and without activation of the inhaler device. As may be seen on FIG. 3A, different frequency peaks having different magnitudes are present for different inhalation flows.

FIG. 3B is a graph (such as spectrogram) representing the operation of FIG. 3A where the audio signal has been transformed to a spectrogram by the processor such as processor 10C of FIG. 1), with the frequency (such as sound frequency) in Hz on the Y-axis and time in s (seconds) on the X-axis. The amplitude in dB is represented with the color-coded scale on the right side of the graph.

FIGS. 3A-3B allow to visualize the relationship between inhalation flow rate, sound frequency profile, and sound amplitude for inhalations using an inhaler device as disclosed herein. Operation data as shown in FIGS. 3A-3B may be used as input to the predictor circuitry, such as to the prediction model as disclosed herein for determining a predicted operation parameter as disclosed herein. It may be appreciated that the operation data as shown in FIGS. 3A-3B may be used to train the prediction model as disclosed herein. The electronic device has determined based on the operation data of FIGS. 3A-3B an predicted operation parameter comprising an inhaler device status parameter being indicative of a type of inhaler device used in the operation data. The type of inhaler device was determined to be a metered-type inhaler device.

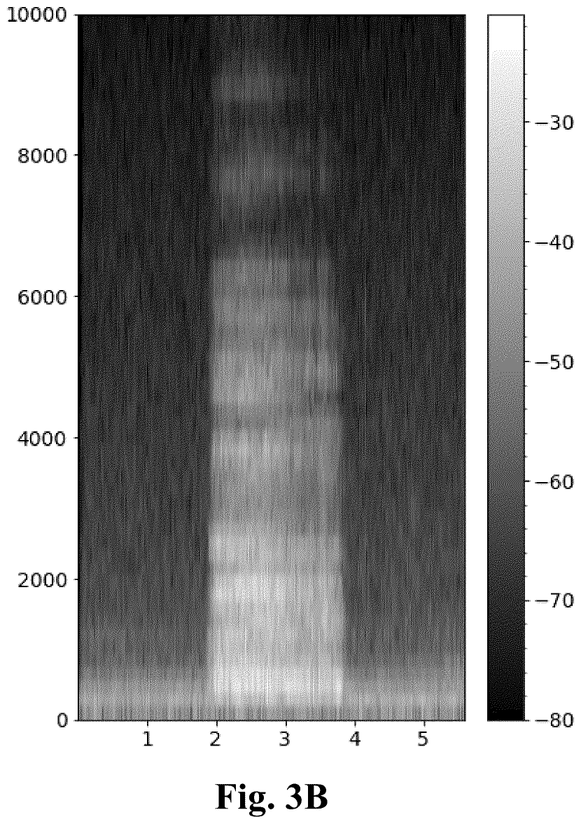

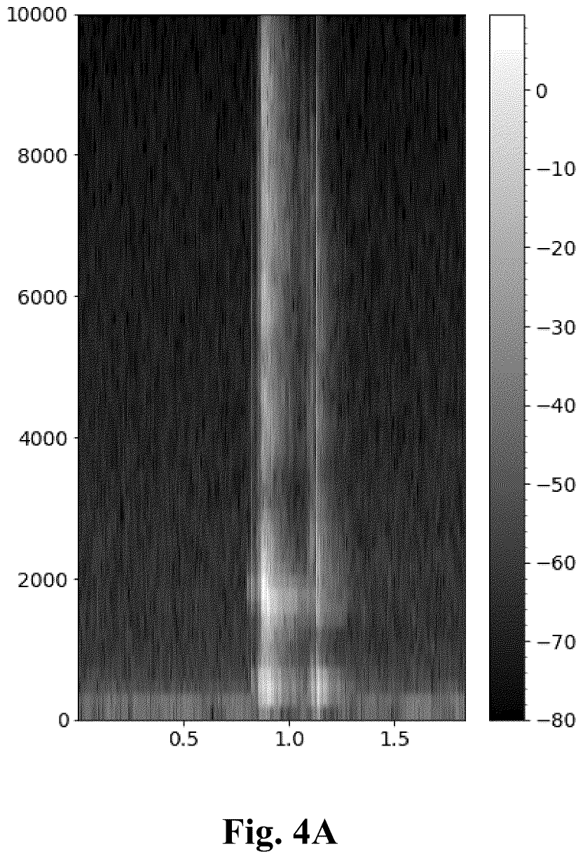

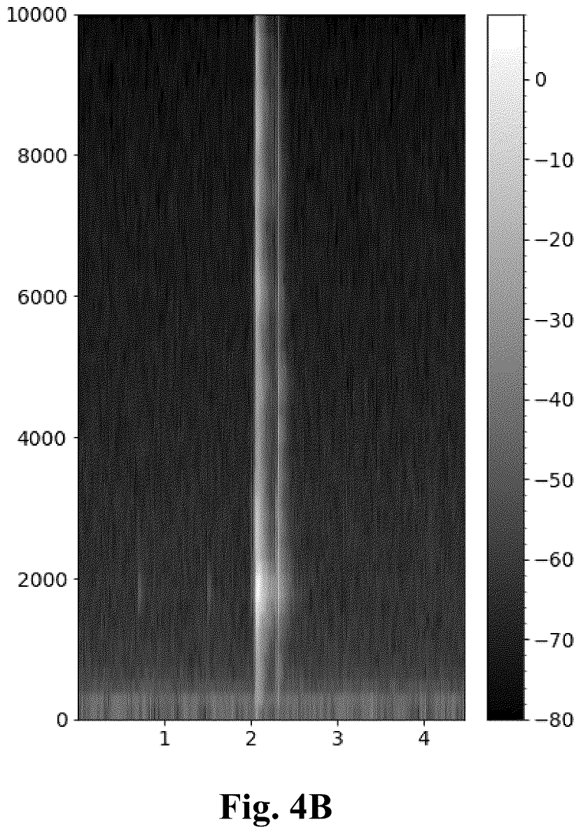

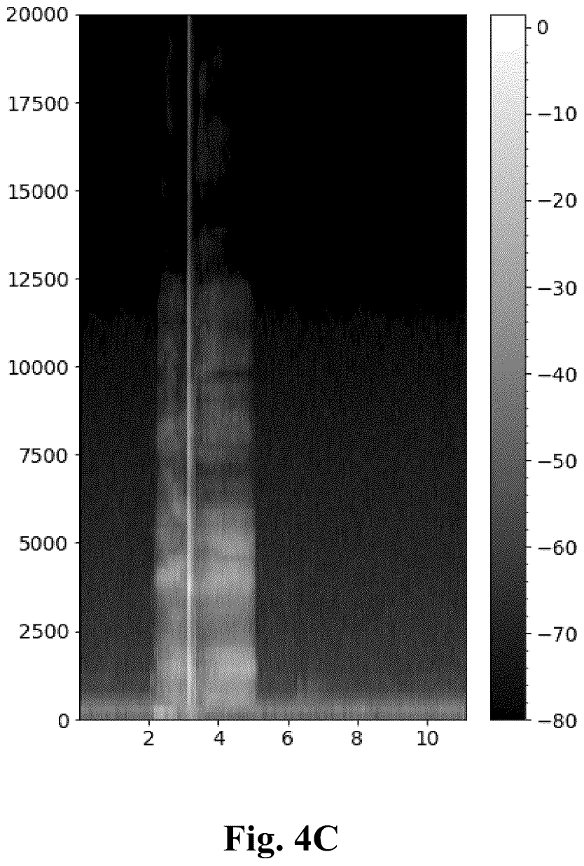

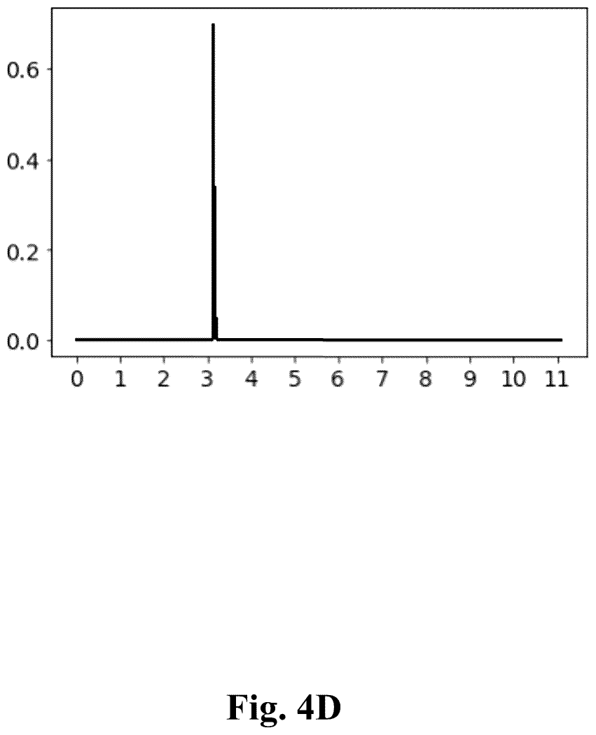

FIGS. 4A-4D show an example scenario of characterization and/or monitoring of an operation of an inhaler device where the technique disclosed herein is applied. In other words, FIGS. 4A-4D show an example scenario where an electronic device according to the present disclosure has been used to characterize and/or monitor an operation of an inhaler device.

FIGS. 4A-4C show a visualization (such as representation) of operation data indicative of an audio signal representing an operation of the inhaler device. The operation data is in FIG. 4A visualized (such as represented) in the form of a spectrogram of an activation sound profile representing an activation (such as an actuation) of an inhaler device as disclosed herein. The inhaler device is of the metered-type inhaler device. The activation represented in FIG. 4A has been performed while the inhaler device was in the mouth of the user. The operation data is in FIG. 4B visualized (such as represented) in the form of a spectrogram of an activation sound profile representing an activation (such as an actuation) of an inhaler device as disclosed herein. The inhaler device is of the metered-type inhaler device. The activation represented in FIG. 4B has been performed while the inhaler device was in the air and not in mouth of the user. As may be seen when comparing FIGS. 4A and 4B an activation of the inhaler device in the mouth of the user and in the air results in a different spectrogram. The electronic device is capable of identifying this difference, and to determine a predicted operation parameter based on this difference.

The operation data is in FIG. 4C visualized (such as represented) in the form of a spectrogram of an activation sound profile representing an activation (such as an actuation) of an inhaler device as disclosed herein and an inhalation sound profile of an inhalation performed with the inhaler. The inhaler device is of the metered-type inhaler device. In other words, the operation in FIG. 4C represents at least two operations in the form of an inhalation and an activation of the inhaler device. As it may be seen on FIG. 4C, the operation indicative of an activation and the operation of an inhalation may be distinguishable by the electronic device.

The operation data has been obtained by the processor (such as processor 10C of FIG. 1).

FIGS. 4A-4C show an example representation of operation data indicative of an audio signal representing an operation (such as one or more operations) with an inhaler device as disclosed herein. FIGS. 4A-4C are spectrograms representing the frequency (such as sound frequency) in Hz on the Y-axis and time in s (seconds) on the X-axis. The amplitude in dB is represented with the color-coded scale on the right side of the graphs. Operation data as shown in FIGS. 4A-4C may be used as input to the predictor circuitry, such as to the prediction model as disclosed herein for determining a predicted operation parameter as disclosed herein. It may be appreciated that the operation data as shown in FIGS. 4A-4C may be used to train the prediction model as disclosed herein.

The electronic device has determined a predicted operation parameter based on the operation data of FIG. 4C. The electronic device has determined an operation representation based on the predicted operation parameter which is shown in FIG. 4D. The representation of FIG. 4D may be outputted via the interface of the electronic device, e.g., outputted to the user of the electronic device and/or inhaler device. FIG. 4D is a graph representing the determined operation representation. The electronic device has determined based on the operation data of one or more of FIGS. 4A-4C a predicted operation parameter. The graph of FIG. 4D represents on the Y-axis the predicted operation parameter indicative of a prediction of an operation with the inhaler device. The predicted operation parameter on the Y-axis is indicative of a probability that an activation took place at each point in time. As may be seen the electronic device has determined a probability of 0.7 that an activation has taken at 3.1 s.

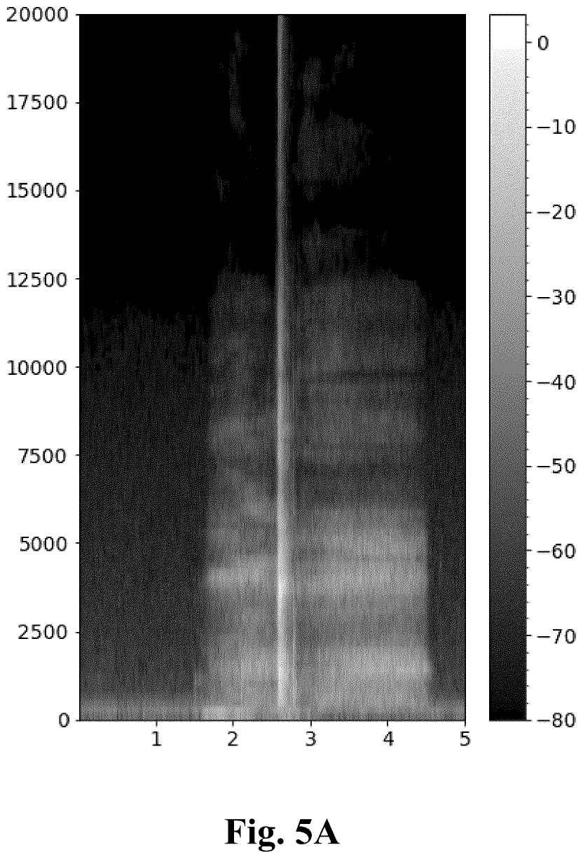

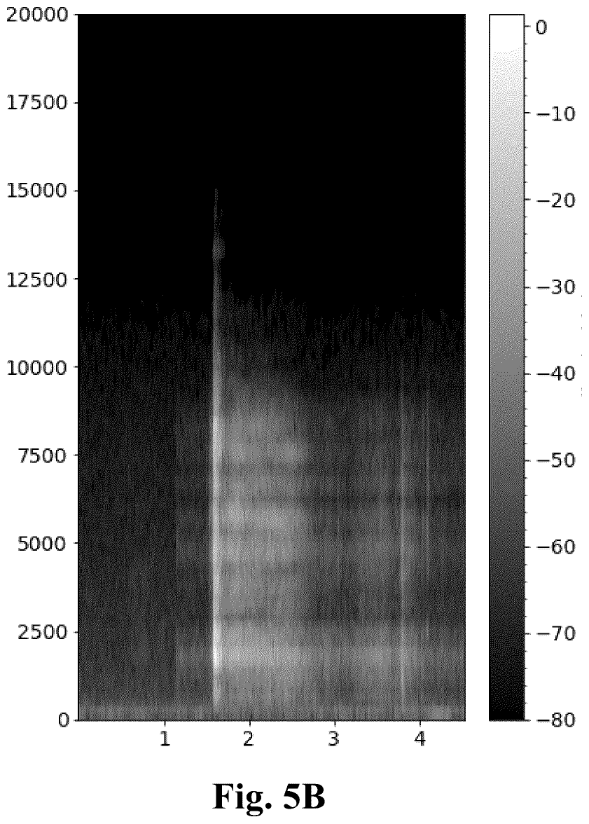

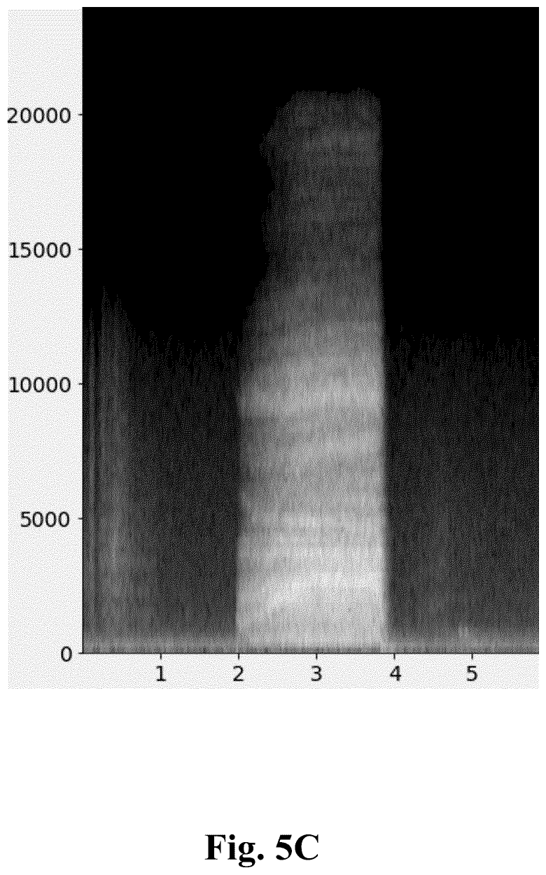

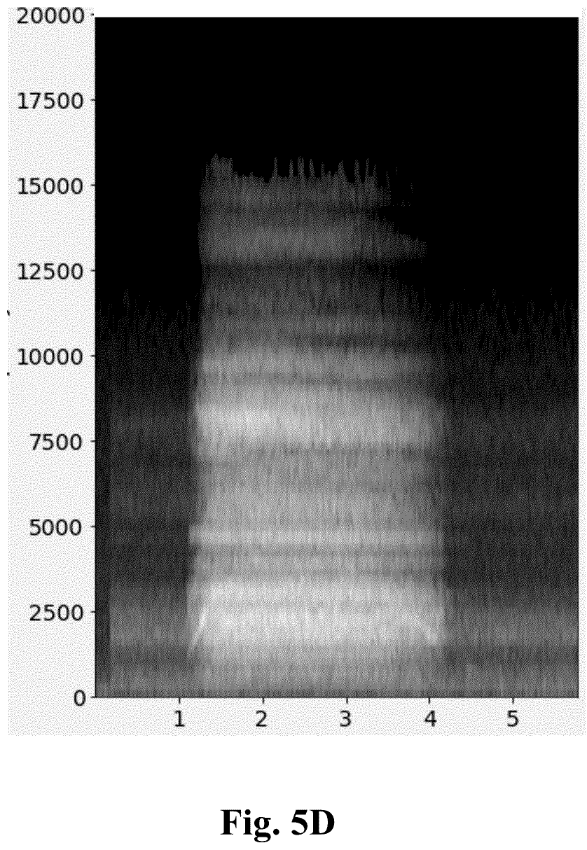

FIGS. 5A-5D show an example scenario of characterization and/or monitoring of an operation of an inhaler device where the technique disclosed herein is applied. In other words, FIGS. 5A-5D show an example scenario where an electronic device according to the present disclosure has been used to characterize and/or monitor an operation of an inhaler device.

FIGS. 5A-5D show a visualization (such as representation) of operation data indicative of an audio signal representing an operation of the inhaler device.

The operation data is in FIGS. 5A-5D visualized (such as represented) in the form of spectrograms of inhalation sound profiles representing an inhalation with different inhaler devices as disclosed herein. The operation data has been obtained by the processor (such as processor 10C of FIG. 1).

FIGS. 5A-5D show an example representation of operation data indicative of an audio signal representing an operation (such as one or more operations) with four different inhaler devices.

FIGS. 5A-5D are spectrograms representing the frequency (such as sound frequency) in Hz on the Y-axis and time in s (seconds) on the X-axis. The amplitude in dB is represented with the color-coded scale on the right side of the graphs. Operation data as shown in FIGS. 5A-5D may be used as input to the predictor circuitry, such as to the prediction model as disclosed herein for determining a predicted operation parameter as disclosed herein. It may be appreciated that the operation data as shown in FIGS. 5A-5D may be used to train the prediction model as disclosed herein.

FIG. 5A is a graph (such as spectrogram) representing the operation of an inhalation with a first type of inhaler device of metered-type inhaler device.

FIG. 5B is a graph (such as spectrogram) representing the operation of an inhalation with a second type of inhaler device of metered-type inhaler device. As may be seen by comparing the spectrogram of FIG. 5A and the spectrogram of FIG. 5B, the inhalation sound profile is different for two different types of metered-type inhaler devices.

FIG. 5C is a graph (such as spectrogram) representing the operation of an inhalation with a first type of inhaler device of dry-powdered-type inhaler device.

FIG. 5D is a graph (such as spectrogram) representing the operation of an inhalation with a second type of inhaler device of dry-powdered-type inhaler device. As may be seen by comparing the spectrogram of FIG. 5C and the spectrogram of FIG. 5D, the inhalation sound profile is different for two different types of dry-powdered-type inhaler devices.

The electronic device has determined based on the operation data of any one or more of FIGS. 5A-5D a predicted operation parameter comprising an inhaler device status parameter being indicative of a type of inhaler device used in the operation data for each inhalation of FIGS. 5A-5D. For the operation data of FIG. 5A, the type of inhaler device was determined to be a first type of metered-type inhaler device. For the operation data of FIG. 5B, the type of inhaler device was determined to be a second type of metered-type inhaler device. For the operation data of FIG. 5C, the type of inhaler device was determined to be a first type of dry-powdered-type inhaler device. For the operation data of FIG. 5D, the type of inhaler device was determined to be a second type of dry-powdered-type inhaler device.

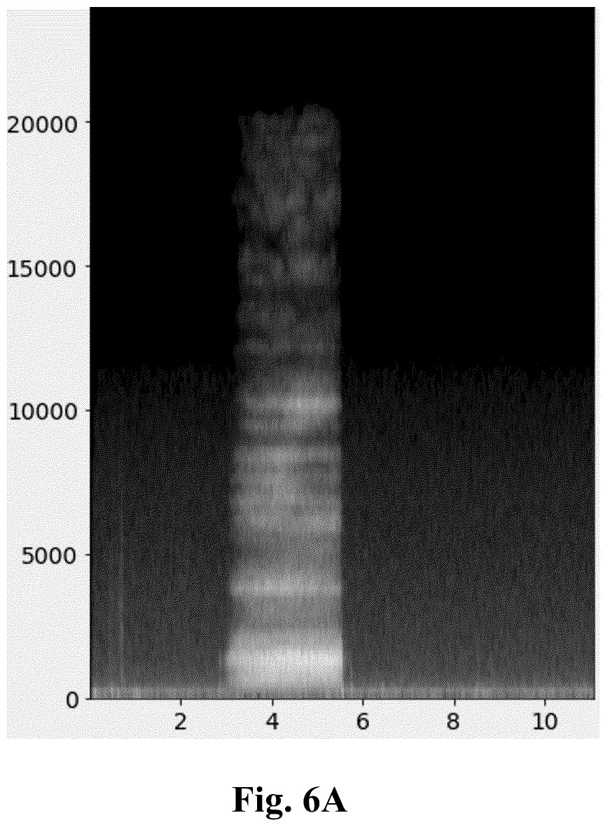

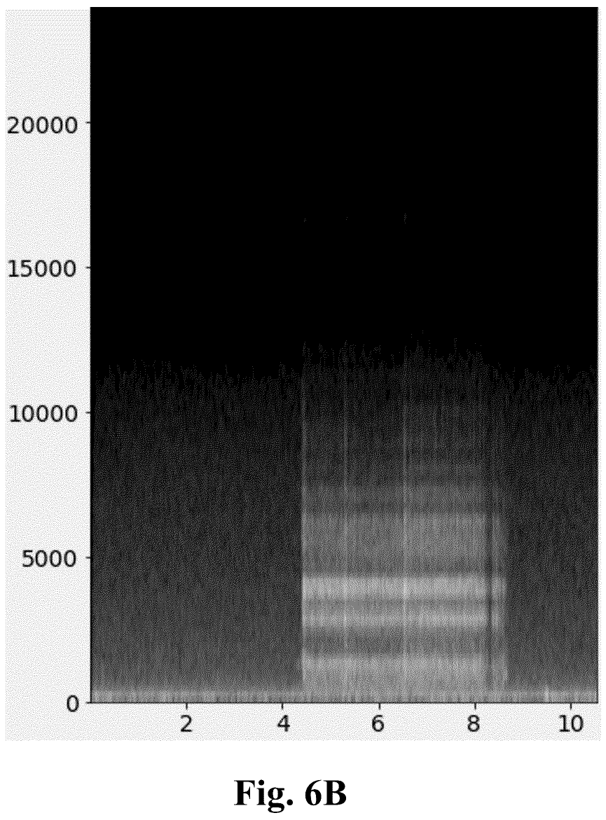

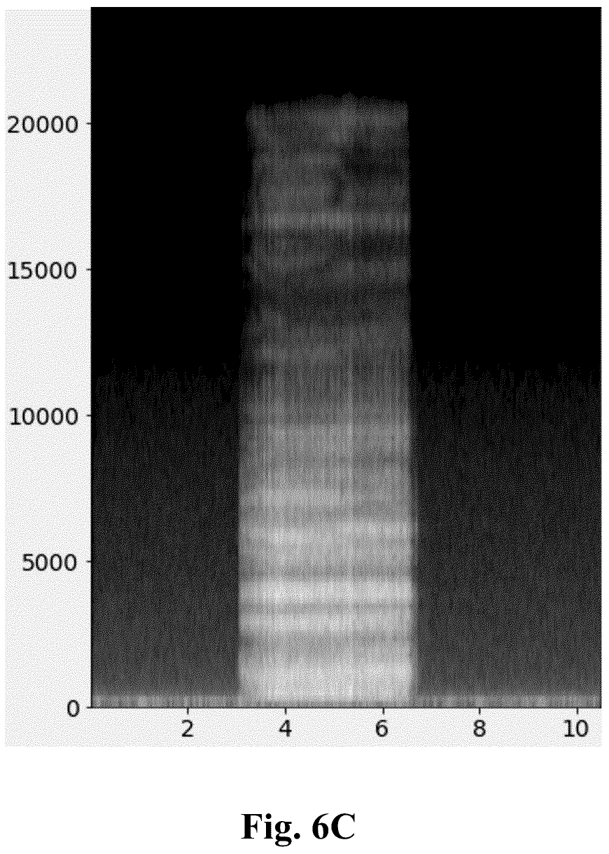

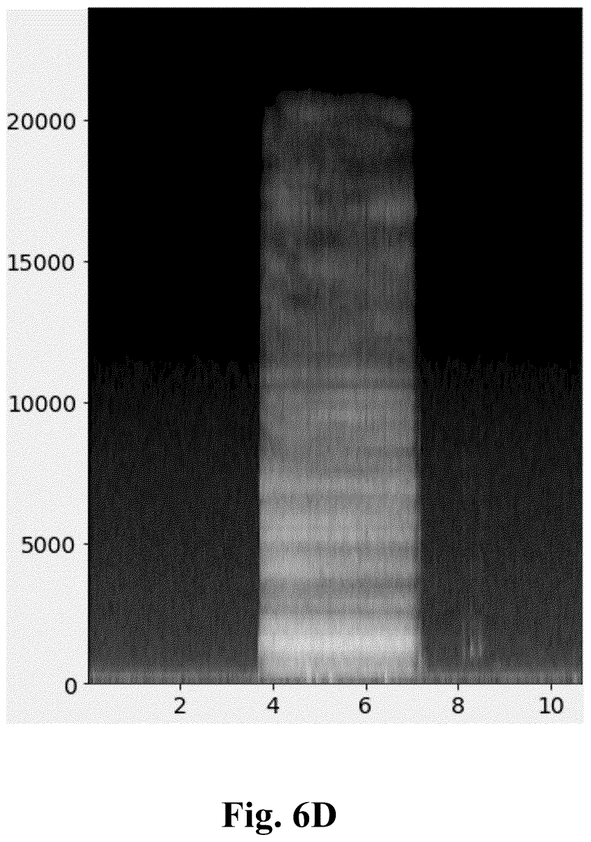

FIGS. 6A-6D show an example scenario of characterization and/or monitoring of an operation of an inhaler device where the technique disclosed herein is applied. In other words, FIGS. 6A-6D show an example scenario where an electronic device according to the present disclosure has been used to characterize and/or monitor an operation of an inhaler device.

FIGS. 6A-6D show a visualization (such as representation) of operation data indicative of an audio signal representing an operation of the inhaler device.

The operation data is in FIGS. 6A-6D visualized (such as represented) in the form of spectrograms of inhalation sound profiles representing inhalations with the same inhaler device but with containers having different container status. The operation data has been obtained by the processor (such as processor 10C of FIG. 1).

FIGS. 6A-6D show an example representation of operation data indicative of an audio signal representing an operation (such as one or more operations) with four different container statuses.

FIGS. 6A-6D are spectrograms representing the frequency (such as sound frequency) in Hz on the Y-axis and time in s (seconds) on the X-axis. Operation data as shown in FIGS. 6A-6D may be used as input to the predictor circuitry, such as to the prediction model as disclosed herein for determining a predicted operation parameter as disclosed herein. It may be appreciated that the operation data as shown in FIGS. 6A-6D may be used to train the prediction model as disclosed herein.

FIG. 6A is a graph (such as spectrogram) representing the operation of an inhalation with a first container status. FIG. 6B is a graph (such as spectrogram) representing the operation of an inhalation with a second container status. FIG. 6C is a graph (such as spectrogram) representing the operation of an inhalation with a third container status. FIG. 6D is a graph (such as spectrogram) representing the operation of an inhalation with a fourth container status.

As may be seen by comparing the spectrograms of FIGS. 6A-6D, the inhalation sound profiles are different for four different inhalations with four different types of container statuses with the same inhaler device.

The electronic device has determined based on the operation data of any one or more of FIGS. 6A-6D a predicted operation parameter comprising a container status parameter being indicative of a container status of the inhaler device used in the operation data for each inhalation of FIGS. 6A-6D. For the operation data of FIG. 6A, the predicted operation parameter was determined to comprise a container status parameter indicative of an inhalation without container (e.g., no container present in the inhaler device), such as with no capsule being mounted. For the operation data of FIG. 6B, the predicted operation parameter was determined to comprise a container status parameter indicative of an inhalation with a container having a container content being full (such as with a full capsule) and with a low inhalation flow. For the operation data of FIG. 6C, the predicted operation parameter was determined to comprise a container status parameter indicative of an inhalation with a container having a container content being full (such as with a full capsule) and with a high inhalation flow. Further, the predicted operation parameter was determined to comprise a container status parameter indicative of a rotating capsule. For the operation data of FIG. 6D, the predicted operation parameter was determined to comprise a container status parameter indicative of an inhalation with a container having a container content being empty (such as with an empty capsule) and with a high inhalation flow. Further, the predicted operation parameter was determined to comprise a container status parameter indicative of a rotating capsule. The type of inhaler device for the operation data of FIGS. 6A-6D was determined to be a dry-powdered-type inhaler device.

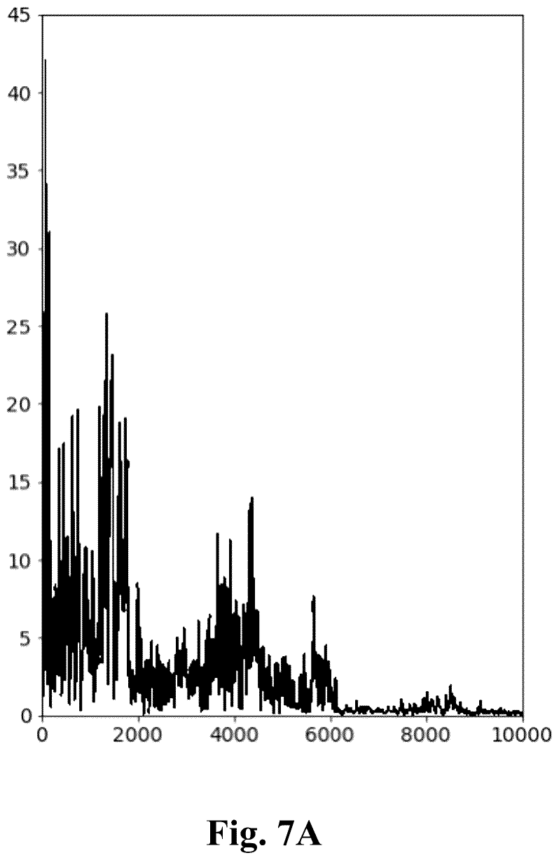

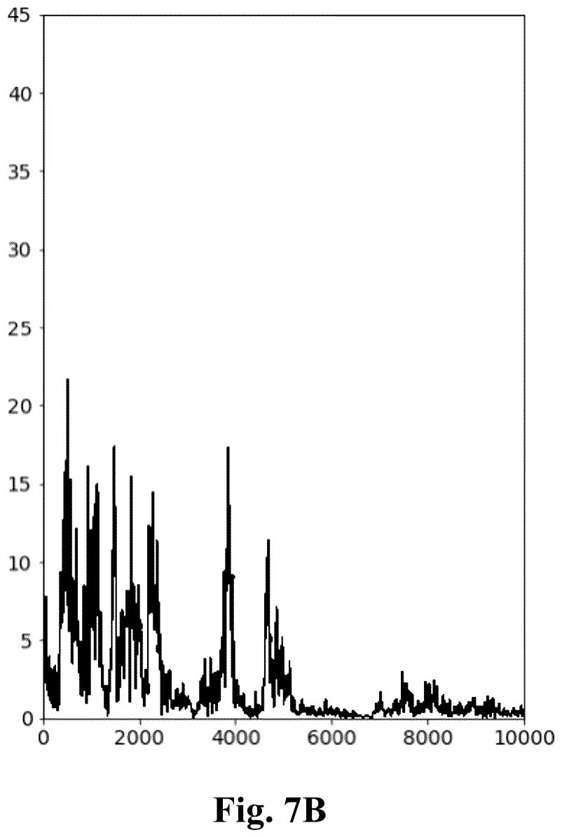

FIGS. 7A-7B show an example scenario of characterization and/or monitoring of an operation of an inhaler device where the technique disclosed herein is applied. In other words, FIGS. 7A-7B show an example scenario where an electronic device according to the present disclosure has been used to characterize and/or monitor an operation of an inhaler device.

FIGS. 7A-7B show a visualization (such as representation) of operation data indicative of an audio signal representing an operation of the inhaler device. FIG. 7A is a graph representing frequency (such as sound frequency) in Hz on the X-axis and magnitude (such as energy) on the Y-axis. The magnitude is a relative difference in magnitude and is therefore unitless. The graph of FIG. 7A represents the magnitude with respect to frequency for an inhalation performed with an inhaler device of a metered dose inhaler type without acoustic amplifier and with activation of the inhaler device. The graph of FIG. 7A represents the magnitude with respect to frequency for an inhalation performed with an inhaler device comprising a full container (such as a full canister). FIG. 7B is a graph representing frequency (such as sound frequency) in Hz on the X-axis and magnitude (such as energy) on the Y-axis. The magnitude is a relative difference in magnitude and is therefore unitless. The graph of FIG. 7B represents the magnitude with respect to frequency for an inhalation performed with an inhaler device of a metered dose inhaler type without acoustic amplifier and with activation of the inhaler device. The graph of FIG. 7B represents the magnitude with respect to frequency for an inhalation performed with an inhaler device comprising an empty container (such as an empty canister).

Operation data as shown in FIGS. 7A-7B may be used as input to the predictor circuitry, such as to the prediction model as disclosed herein for determining a predicted operation parameter as disclosed herein. It may be appreciated that the operation data as shown in FIGS. 7A-7B may be used to train the prediction model as disclosed herein.

The electronic device has determined an operation representation based on the predicted operation parameter which is shown in FIG. 7A and FIG. 7B. The electronic device has determined based on the operation data of any one or more of FIGS. 7A-7B a predicted operation parameter comprising a container status parameter being indicative of a container status of the inhaler device used in the operation data for each inhalation of FIGS. 7A-7B. For the operation data of FIG. 7A, the predicted operation parameter was determined to comprise a container status parameter indicative of an inhalation with a container having a container content being full (such as with a full canister). For the operation data of FIG. 7B, the predicted operation parameter was determined to comprise a container status parameter indicative of an inhalation with a container having a container content being empty (such as with an empty canister).

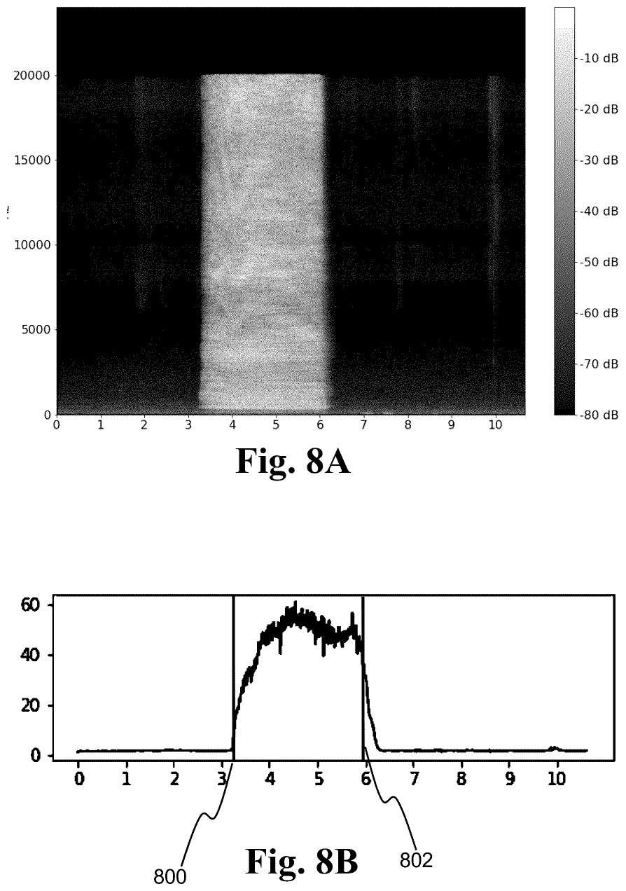

FIGS. 8A-8B show an example scenario of characterization and/or monitoring of an operation of an inhaler device where the technique disclosed herein is applied. In other words, FIGS. 8A-8B show an example scenario where an electronic device according to the present disclosure has been used to characterize and/or monitor an operation of an inhaler device.

FIG. 8A shows a visualization (such as representation) of operation data indicative of an audio signal representing an operation of the inhaler device. FIG. 8A is a graph representing an inhalation with an inhaler device of a multi-dose dry powder inhaler type without acoustic amplifier as disclosed herein where the frequency (such as sound frequency) in Hz is on the Y-axis and the time in seconds (s) is on the X-axis. The predicted operation parameter is therefore merely determine based on inhalation sound and not acoustically amplified inhalation sound. The operation data is in FIG. 8A visualized (such as represented) in the form of a plot of an inhalation sound profile. The operation data has been obtained by the processor (such as processor 10C of FIG. 1).

Operation data as shown in FIG. 8A may be used as input to the predictor circuitry, such as to the prediction model as disclosed herein for determining a predicted operation parameter as disclosed herein. It may be appreciated that the operation data as shown in FIG. 8A may be used to train the prediction model as disclosed herein.

The processor of the electronic device has determined, based on the operation data represented in FIG. 8A, using the predictor circuitry, a predicted operation parameter indicative of a prediction of an operation of the inhaler device. The processor of the electronic device has determined, based on the predicted operation parameter, an operation representation. The operation representation is shown in FIG. 8B. The representation of FIG. 8B may be outputted via the interface of the electronic device, e.g., outputted to the user of the electronic device and/or inhaler device. FIG. 8B is a graph representing the determined operation representation. The graph of FIG. 8B represents on the Y-axis the predicted operation parameter indicative of a prediction of an inhalation flow with the inhaler device in I/min. In other words, the electronic device has determined that the predicted operation parameter is indicative of an inhalation. The electronic device has determined, based on the predicted operation parameter, a duration of inhalation. As may be seen on FIG. 8B, the beginning 800 of the inhalation (such as an introductory inhalation phase) has been determined by the processor and the ending 802 of the inhalation (such as an ending phase) has been determined by the processor. The duration of the inhalation has been determined, based on the beginning 800 and ending 802 of the inhalation, to be approximately 3 s.

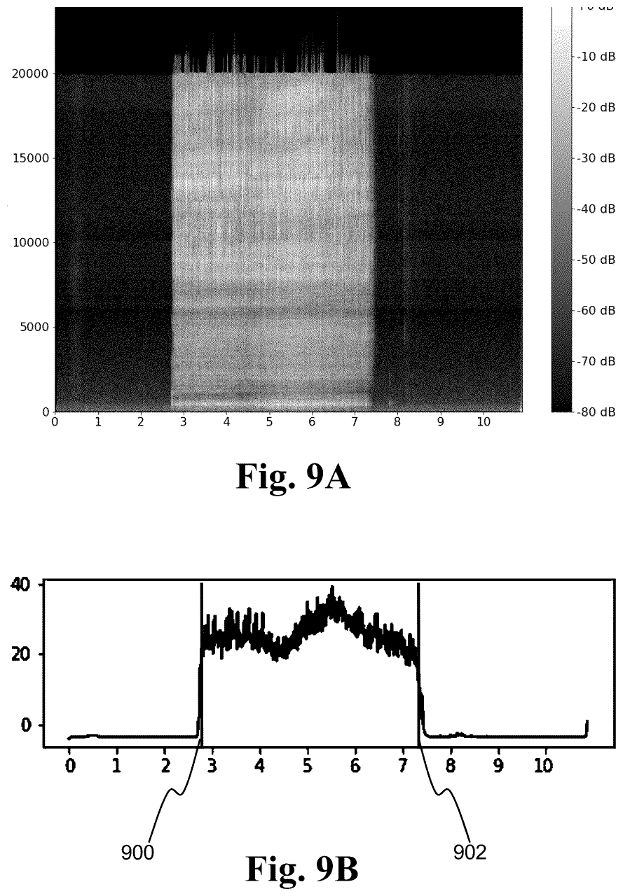

FIGS. 9A-9B show an example scenario of characterization and/or monitoring of an operation of an inhaler device where the technique disclosed herein is applied.

FIGS. 9A-9B show an example scenario of characterization and/or monitoring of an operation of an inhaler device where the technique disclosed herein is applied. In other words, FIGS. 9A-9B show an example scenario where an electronic device according to the present disclosure has been used to characterize and/or monitor an operation of an inhaler device.

FIG. 9A shows a visualization (such as representation) of operation data indicative of an audio signal representing an operation of the inhaler device. FIG. 9A is a graph representing an inhalation with an inhaler device of a capsule-based dry powder inhaler type without acoustic amplifier as disclosed herein where the frequency (such as sound frequency) in Hz is on the Y-axis and the time in seconds (s) is on the X-axis. The predicted operation parameter is determined based on the sound of the capsule moving (such as rotating in the inhaler device). The operation data is in FIG. 9A visualized (such as represented) in the form of a plot of an inhalation sound profile. The operation data has been obtained by the processor (such as processor 10C of FIG. 1).

Operation data as shown in FIG. 9A may be used as input to the predictor circuitry, such as to the prediction model as disclosed herein for determining a predicted operation parameter as disclosed herein. It may be appreciated that the operation data as shown in FIG. 9A may be used to train the prediction model as disclosed herein.

The processor of the electronic device has determined, based on the operation data represented in FIG. 9A, using the predictor circuitry, a predicted operation parameter indicative of a prediction of an operation of the inhaler device. The processor of the electronic device has determined, based on the predicted operation parameter, an operation representation. The operation representation is shown in FIG. 9B. The representation of FIG. 9B may be outputted via the interface of the electronic device, e.g., outputted to the user of the electronic device and/or inhaler device. FIG. 9B is a graph representing the determined operation representation. The graph of FIG. 9B represents on the Y-axis the predicted operation parameter indicative of a prediction of an inhalation flow with the inhaler device in I/min. In other words, the electronic device has determined that the predicted operation parameter is indicative of an inhalation. The electronic device has determined, based on the predicted operation parameter, a duration of inhalation. As may be seen on FIG. 9B, the beginning 900 of the inhalation (such as an introductory inhalation phase) has been determined by the processor and the ending 902 of the inhalation (such as an ending phase) has been determined by the processor. The duration of the inhalation has been determined, based on the beginning 900 and ending 902 of the inhalation, to be approximately 4.5 s.

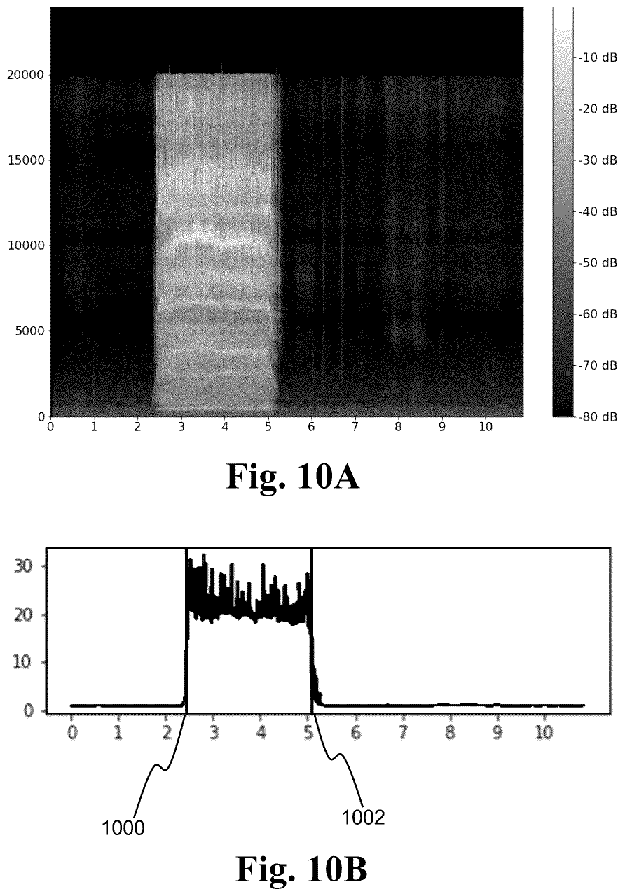

FIGS. 10A-10B show an example scenario of characterization and/or monitoring of an operation of an inhaler device where the technique disclosed herein is applied.

FIGS. 10A-10B show an example scenario of characterization and/or monitoring of an operation of an inhaler device where the technique disclosed herein is applied. In other words, FIGS. 10A-10B show an example scenario where an electronic device according to the present disclosure has been used to characterize and/or monitor an operation of an inhaler device.

FIG. 10A shows a visualization (such as representation) of operation data indicative of an audio signal representing an operation of the inhaler device. FIG. 10A is a graph representing an inhalation with an inhaler device of a capsule-based dry powder inhaler type and with acoustic amplifier as disclosed herein where the frequency (such as sound frequency) in Hz is on the Y-axis and the time in seconds (s) is on the X-axis. The predicted operation parameter is determined based on the sound of the capsule moving (such as rotating in the inhaler device) and the amplified inhalation sound from the acoustic amplifier. The operation data is in FIG. 10A visualized (such as represented) in the form of a plot of an inhalation sound profile. The operation data has been obtained by the processor (such as processor 10C of FIG. 1).

Operation data as shown in FIG. 10A may be used as input to the predictor circuitry, such as to the prediction model as disclosed herein for determining a predicted operation parameter as disclosed herein. It may be appreciated that the operation data as shown in FIG. 10A may be used to train the prediction model as disclosed herein.

The processor of the electronic device has determined, based on the operation data represented in FIG. 10A, using the predictor circuitry, a predicted operation parameter indicative of a prediction of an operation of the inhaler device. The processor of the electronic device has determined, based on the predicted operation parameter, an operation representation. The operation representation is shown in FIG. 10B. The representation of FIG. 10B may be outputted via the interface of the electronic device, e.g., outputted to the user of the electronic device and/or inhaler device. FIG. 10B is a graph representing the determined operation representation. The graph of FIG. 10B represents on the Y-axis the predicted operation parameter indicative of a prediction of an inhalation flow with the inhaler device in I/min. In other words, the electronic device has determined that the predicted operation parameter is indicative of an inhalation. The electronic device has determined, based on the predicted operation parameter, a duration of inhalation. As may be seen on FIG. 10B, the beginning 1000 of the inhalation (such as an introductory inhalation phase) has been determined by the processor and the ending 1002 of the inhalation (such as an ending phase) has been determined by the processor. The duration of the inhalation has been determined, based on the beginning 1000 and ending 1002 of the inhalation, to be approximately 2.7 s.

As may be seen on FIGS. 10A-10B, the sound profile for a capsule-based inhaler with an acoustic amplifier is very different from the sound profiles of e.g., FIGS. 8A-8B that are based on inhalation data performed with a metered dose type inhaler. The inhalation flow prediction demonstrated that even at low inhalation flow rates the flow rate could be predicted.

Examples of electronic devices, systems, and methods according to the disclosure are set out in the following items:

Item 1. An electronic device for characterizing and/or monitoring an operation of an inhaler device, the electronic device comprising:
a memory;
an interface;
and a processor comprising predictor circuitry configured to operate according to a prediction model, wherein the processor is configured to:
obtain operation data, where the operation data is indicative of an audio signal representing an operation of the inhaler device;
determine, based on the operation data, using the predictor circuitry, a predicted operation parameter indicative of a prediction of an operation of the inhaler device;
determine, based on the predicted operation parameter, an operation representation; and
output, via the interface, the operation representation.

Item 2. Electronic device according to item 1, wherein the determination of the predicted operation parameter comprises to determine one or more of: an inhalation parameter, an inhaler device status parameter, an activation parameter, a coordination parameter, and a container status parameter.

Item 3. Electronic device according to item 2, wherein the determination of the container status parameter comprises to determine whether a container is present in the inhaler device.

Item 4. Electronic device according to any of items 2-3, wherein the determination of the container status parameter comprises to determine a content of a container of the inhaler device.

Item 5. Electronic device according to any of the previous items, wherein the processor is configured to determine, based on the predicted operation parameter, one or more of: a duration of the operation, a coordination of the operation, an inhalation volume, an average inhalation flow, a maximum inhalation flow, a minimum inhalation flow, median inhalation flow, an inhalation flow acceleration, an inhalation pattern, and one or more exhalation parameters.

Item 6. Electronic device according to any of the previous items, wherein the operation representation is indicative of a performance of the operation of the inhaler device.

Item 7. Electronic device according to any of the previous items, wherein the processor is configured to:
determine whether the predicted operation parameter satisfies a first criterion;
in accordance with the predicted operation parameter satisfying the first criterion, determine a first recommendation; and
output, via the interface, the first recommendation.

Item 8. Electronic device according to item 7, wherein the first criterion comprises one or more of: a coordination criterion, an activation criterion, an inhaler status criterion, inhalation criterion, and a container status criterion.

Item 9. Electronic device according to any of items 7-8, wherein the first recommendation is comprised in the operation representation.

Item 10. Electronic device according to any of items 7-8, wherein the first recommendation comprises one or more of: an inhaler device maintenance recommendation, a container recommendation, a coordination recommendation, an inhalation depth recommendation, an inhalation duration recommendation, an inhalation flow rate recommendation, and an inhalation preparation recommendation.

Item 11. Electronic device according to any of the previous items, wherein the electronic device comprises one or more microphones for obtaining the audio signal.

Item 12. Electronic device according to any of the previous items, wherein the obtaining of the operation data comprises to obtain, based on the audio signal, sound data of the audio signal, and wherein the determination of the predicted operation parameter is based on the sound data.

Item 13. Electronic device according to item 12, wherein the sound data comprises one or more of: a frequency signature, an amplitude signature, and a duration signature.

Item 14. Electronic device according to any of the previous items, wherein the obtaining of the operation data comprises to perform pre-processing of the audio signal.

Item 15. Electronic device according to any of the previous items, wherein the obtaining of the operation data comprises to identify a background noise from the audio signal.

Item 16. Electronic device according to any of the previous items, wherein the predictor circuitry comprises a neural network module configured to operate according to a neural network.

Item 17. Electronic device according to item 16, wherein the neural network is a deep neural network, such as a classification neural network configured to operate according to a classification model.

Item 18. Electronic device according to any of the previous items, wherein the obtaining of operation data comprises to split the audio signal into a plurality of audio samples and wherein the determination of the predicted operation parameter is based on one or more audio samples of the plurality of audio samples.

Item 19. Electronic device according to item 18, wherein the obtaining of operation data comprises to shuffle the plurality of audio samples and wherein the determination of the predicted operation parameter is based on the shuffled audio samples.

Item 20. Electronic device according to any of the previous items, wherein the obtaining of operation data comprises to transform the audio signal to a spectrogram, and wherein the determination of the predicted operation parameter is based on the spectrogram.

Item 21. Electronic device according to any of the previous items, wherein the electronic device is a user equipment device.

Item 22. Electronic device according to any of the previous items, wherein the electronic device is a server device.

Item 23. A system for characterizing and/or monitoring an operation of an inhaler device, the system comprising the inhaler device and an electronic device according to any of items 1-22.

Item 24. A method for characterizing and/or monitoring an operation of an inhaler device, the method comprising:
obtaining (S102) operation data, where the operation data is indicative of an audio signal representing an operation of the inhaler device;
determining (S104), based on the operation data, a predicted operation parameter indicative of a prediction of an operation of the inhaler device;
determining (S106), based on the predicted operation parameter, an operation representation; and
outputting (S118) the operation representation.

Item 25. The method according to item 24, wherein the determination (S104) of the predicted operation parameter comprises:
determining (S104A) one or more of: an inhalation parameter, an inhaler device status parameter, an activation parameter, a coordination parameter, and a container status parameter.

Item 26. The method according to item 25, wherein the determination of the container status parameter comprises:
determining (S108) whether a container is present in the inhaler device.

Item 27. The method according to any of items 25-26, wherein the determination of the container status parameter comprises:
determining (S110) a content of a container of the inhaler device.

Item 28. The method according to any of items 24-27, wherein the method comprises:
determining (S112), based on the predicted operation parameter, one or more of: a duration of the operation, a coordination of the operation, an inhalation volume, an average inhalation flow, a maximum inhalation flow, a minimum inhalation flow, a median inhalation flow, an inhalation flow acceleration, an inhalation pattern, and one or more exhalation parameters.

Item 29. The method according to any of items 24-28, wherein the method comprises:
determining (S114) whether the predicted operation parameter satisfies a first criterion;
in accordance with the predicted operation parameter satisfying the first criterion, determining (S116) a first recommendation; and
outputting (S120), the first recommendation.

Item 30. The method according to any of items 24-29, wherein the obtaining (S102) of the operation data comprises:
obtaining (S102A), based on the audio signal, sound data of the audio signal, and wherein the determination (S104) of the predicted operation parameter is based on the sound data.

Item 31. The method according to any of items 24-30, wherein the obtaining (S102) of the operation data comprises:
performing (S102B) pre-processing of the audio signal.

Item 32. The method according to any of items 24-31, wherein the obtaining (S102) of the operation data comprises:
identifying (S102C) a background noise from the audio signal.

Item 33. The method according to any of items 24-32, wherein the obtaining (S102) of the operation data comprises:
splitting (S102D) the audio signal into a plurality of audio samples and wherein the determination (S104) of the predicted operation parameter is based on one or more audio samples of the plurality of audio samples.

Item 34. The method according to item 33, wherein the obtaining (S102) of the operation data comprises:
shuffling (S102E) the plurality of audio samples and wherein the determination (S104) of the predicted operation parameter is based on the shuffled audio samples.

Item 35. The method according to any of items 24-34, wherein the obtaining (S102) of the operation data comprises:
transforming (S102F) the audio signal to a spectrogram, and wherein the determination (S104) of the predicted operation parameter is based on the spectrogram.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that the Figures comprise some circuitries or operations which are illustrated with a solid line and some circuitries, components, features, or operations which are illustrated with a dashed line. Circuitries or operations which are comprised in a solid line are circuitries, components, features or operations which are comprised in the broadest example. Circuitries, components, features, or operations which are comprised in a dashed line are examples which may be comprised in, or a part of, or are further circuitries, components, features, or operations which may be taken in addition to circuitries, components, features, or operations of the solid line examples. It should be appreciated that these operations need not be performed in order presented. Furthermore, it should be appreciated that not all of the operations need to be performed. The example operations may be performed in any order and in any combination. It should be appreciated that these operations need not be performed in order presented. Circuitries, components, features, or operations which are comprised in a dashed line may be considered optional.

Other operations that are not described herein can be incorporated in the example operations. For example, one or more additional operations can be performed before, after, simultaneously, or between any of the described operations.

Certain features discussed above as separate implementations can also be implemented in combination as a single implementation. Conversely, features described as a single implementation can also be implemented in multiple implementations separately or in any suitable sub-combination. Moreover, although features may be described above as acting in certain combinations, one or more features from a claimed combination can, in some cases, be excised from the combination, and the combination may be claimed as any sub-combination or variation of any sub-combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the examples may be implemented at least in part by means of both hardware and software, and that several “means”, “units” or “devices” may be represented by the same item of hardware.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents.

### LIST OF REFERENCES

1 user
2 system
4 input
6 operation representation, output
10 electronic device
10A one or more interfaces
10B memory
10C processor
10D one or more microphones
12 predictor circuitry
12A neural network module
12B regressor module
13 control, transmit
14 obtain
20 server device
20A memory
20B one or more interfaces
20C processor(s)
30 inhaler device
32 sound output
100 method for characterizing and/or monitoring an operation with an inhaler device
S102 obtaining operation data
S102A obtaining, based on the audio signal, sound data of the audio signal
S102B performing pre-processing of the audio signal
S102C identifying a background noise from the audio signal
S102D splitting the audio signal into a plurality of audio samples
S102E shuffling the plurality of audio samples
S102F transforming the audio signal to a spectrogram
S104 determining, based on the operation data, a predicted operation parameter indicative of a prediction of an operation of the inhaler device
S104A determining one or more of: an inhalation parameter, an inhaler device status parameter, an activation parameter, a coordination parameter, and a container status parameter
S106 determining, based on the predicted operation parameter, an operation representation
S108 determining whether a container is present in the inhaler device
S109 determining a second recommendation
S110 determining a content of a container of the inhaler device
S112 determining, based on the predicted operation parameter, one or more of: a duration of the operation, a coordination of the operation, an inhalation volume, an average inhalation flow, a maximum inhalation flow, a minimum inhalation flow, a median inhalation flow, an inhalation flow acceleration, an inhalation pattern, and one or more exhalation parameters
S114 determining whether the predicted operation parameter satisfies a first criterion
S116 determining a first recommendation
S118 outputting the operation representation
S120 outputting the first recommendation
S121 outputting the second recommendation

1. An electronic device for at least one of characterizing or monitoring an operation of an inhaler device, the electronic device comprising:
a memory;
an interface; and
a processor including predictor circuitry configured to operate according to a prediction model, wherein the processor is configured to
obtain operation data, wherein the operation data is indicative of an audio signal representing an operation of the inhaler device,
determine, based on the operation data, using the predictor circuitry, a predicted operation parameter indicative of a prediction of an operation of the inhaler device,
determine, based on the predicted operation parameter, an operation representation, and
output, via the interface, the operation representation.

2. The electronic device according to claim 1, wherein the determination of the predicted operation parameter comprises to determine one or more of: an inhalation parameter, an inhaler device status parameter, an activation parameter, a coordination parameter, or a container status parameter.

3. The electronic device according to claim 2, wherein the determination of the container status parameter comprises to determine whether a container is present in the inhaler device.

4. The electronic device according to claim 2, wherein the determination of the container status parameter comprises to determine a content of a container of the inhaler device.

5. The electronic device according to claim 1, wherein the processor is configured to determine, based on the predicted operation parameter, one or more of: a duration of the operation, a coordination of the operation, an inhalation volume, an average inhalation flow, a maximum inhalation flow, a minimum inhalation flow, median inhalation flow, an inhalation flow acceleration, an inhalation pattern, or one or more exhalation parameters.

6. The electronic device according to claim 1, wherein the operation representation is indicative of a performance of the operation of the inhaler device.

7. The electronic device according to claim 1, wherein the processor is configured to:
determine whether the predicted operation parameter satisfies a first criterion;
in accordance with the predicted operation parameter satisfying the first criterion, determine a first recommendation; and
output, via the interface, the first recommendation.

8. The electronic device according to claim 7, wherein the first criterion comprises one or more of: a coordination criterion, an activation criterion, an inhaler status criterion, inhalation criterion, or a container status criterion.

9. The electronic device according to claim 7, wherein the first recommendation is comprised in the operation representation.

10. The electronic device according to claim 7, wherein the first recommendation comprises one or more of: an inhaler device maintenance recommendation, a container recommendation, a coordination recommendation, an inhalation depth recommendation, an inhalation duration recommendation, an inhalation flow rate recommendation, or an inhalation preparation recommendation.

11. The electronic device according to claim 1, wherein the electronic device comprises one or more microphones for obtaining the audio signal.

12. The electronic device according to claim 1, wherein the obtaining of the operation data comprises to obtain, based on the audio signal, sound data of the audio signal, and wherein the determination of the predicted operation parameter is based on the sound data.

13. The electronic device according to claim 12, wherein the sound data comprises one or more of: a frequency signature, an amplitude signature, or a duration signature.

14. The electronic device according to claim 1, wherein the obtaining of the operation data comprises to perform pre-processing of the audio signal.

15. The electronic device according to claim 1, wherein the obtaining of the operation data comprises to identify a background noise from the audio signal.

16. The electronic device according to claim 1, wherein the predictor circuitry comprises a neural network module configured to operate according to a neural network.

17. The electronic device according to claim 16, wherein the neural network is a deep neural network configured to operate according to a classification model.

18. The electronic device according to claim 1, wherein the obtaining of operation data comprises to split the audio signal into a plurality of audio samples, and wherein the determination of the predicted operation parameter is based on one or more audio samples of the plurality of audio samples.

19. The electronic device according to claim 18, wherein the obtaining of operation data comprises to shuffle the plurality of audio samples and wherein the determination of the predicted operation parameter is based on the shuffled audio samples.

20.-23. (canceled)

24. A method for at least one of characterizing or monitoring an operation of an inhaler device, the method comprising:
obtaining operation data, wherein the operation data is indicative of an audio signal representing an operation of the inhaler device;
determining, based on the operation data, a predicted operation parameter indicative of a prediction of an operation of the inhaler device;
determining, based on the predicted operation parameter, an operation representation; and
outputting the operation representation.

25-35. (canceled)
